SEQUENCE LISTING (26 sequences)

SEQ ID NO: 1 - protein, 163 aa, Chlamydomonas reinhardtii
MNTKYNKEFLLYLAGFVDGDGSIIAQIKPNQSYKFKHQLSLAFQVTQKTQRRWFLDKLVDEIGVGYVRDRGSVSDYILSEIKPLHNFLTQLQPFLKLKQKQANLVLKIIWRLPSAKESPDKFLEVCTWVDQIAALNDSKTRKTTSETVRAVLDSLSEKKKSSP

SEQ ID NO: 2 - protein, 9 aa, Chlamydomonas reinhardtii
LAGLIDADG

SEQ ID NO: 3 - DNA, 4627 bp, Homo sapiens
  source 1..4627: mol_type = genomic DNA
atatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagttcctgcctgcctgcctttgctcagactgtttgccccttactgctcttctaggcctcattctaagccccttctccaagttgcctctccttatttctccctgtctgccaaaaaatctttcccagctcactaagtcagtctcacgcagtcactcattaacccaccaatcactgattgtgccggcacatgaatgcaccaggtgttgaagtggaggaattaaaaagtcagatgaggggtgtgcccagaggaagcaccattctagttgggggagcccatctgtcagctgggaaaagtccaaataacttcagattggaatgtgttttaactcagggttgagaaaacagctaccttcaggacaaaagtcagggaagggctctctgaagaaatgctacttgaagataccagccctaccaagggcagggagaggaccctatagaggcctgggacaggagctcaatgagaaaggagaagagcagcaggcatgagttgaatgaaggaggcagggccgggtcacagggccttctaggccatgagagggtagacagtattctaaggacgccagaaagctgttgatcggcttcaagcaggggagggacacctaatttgcttttcttttttttttttttttttttttttttttttgagatggagttttgctcttgttgcccaggctggagtgcaatggtgcatcttggctcactgcaacctccgcctcccaggttcaagtgattctcctgcctcagcctcccgagtagctgagattacaggcacccgccaccatgcctggctaattttttgtatttttagtagagacagggtttcactatgttggccaggctggtctcgaactcctgacctcaggtgatccacccgcttcagcctcccaaagtgctgggattacaggcgtgagccaccacacccggcctgcttttcttaaagatcaatctgagtgctgtacggagagtgggttgtaagccaagagtagaagcagaaagggagcagttgcagcagagagatgatggaggcctgggcagggtggtggcagggaggtaaccaacaccattcaggtttcaaaggtagaaccatgcagggatgagaaagcaaagaggggatcaaggaaggcagctggattttggcctgagcagctgagtcaatgatagtgccgtttactaagaagaaaccaaggaaaaaatttggggtgcagggatcaaaactttttggaacatatgaaagtacgtgtttatactctttatggcccttgtcactatgtatgcctcgctgcctccattggactctagaatgaagccaggcaagagcagggtctatgtgtgatggcacatgtggccagggtcatgcaacatgtactttgtacaaacagtgtatattgagtaaatagaaatggtgtccaggagccgaggtatcggtcctgccagggccaggggctctccctagcaggtgctcatatgctgtaagttccctccagatctctccacaaggaggcatggaaaggctgtagttgttcacctgcccaagaactaggaggtctggggtgggagagtcagcctgctctggatgctgaaagaatgtctgtttttccttttagaaagttcctgtgatgtcaagctggtcgagaaaagctttgaaacaggtaagacaggggtctagcctgggtttgcacaggattgcggaagtgatgaacccgcaataaccctgcctggatgagggagtgggaagaaattagtagatgtgggaatgaatgatgaggaatggaaacagcggttcaagacctgcccagagctgggtggggtctctcctgaatccctctcaccatctctgactttccattctaagcactttgaggatgagtttctagcttcaatagaccaaggactctctcctaggcctctgtattcctttcaacagctccactgtcaagagagccagagagagcttctgggtggcccagctgtgaaatttctgagtcccttagggatagccctaaacgaaccagatcatcctgaggacagccaagaggttttgccttctttcaagacaagcaacagtactcacataggctgtgggcaatggtcctgtctctcaagaatcccctgccactcctcacacccaccctgggcccatattcatttccatttgagttgttcttattgagtcatccttcctgtggtagcggaactcactaaggggcccatctggacccgaggtattgtgatgataaattctgagcacctaccccatccccagaagggctcagaaataaaataagagccaagtctagtcggtgtttcctgtcttgaaacacaatactgttggccctggaagaatgcacagaatctgtttgtaaggggatatgcacagaagctgcaagggacaggaggtgcaggagctgcaggcctcccccacccagcctgctctgccttggggaaaaccgtgggtgtgtcctgcaggccatgcaggcctgggacatgcaagcccataaccgctgtggcctcttggttttacagatacgaacctaaactttcaaaacctgtcagtgattgggttccgaatcctcctcctgaaagtggccgggtttaatctgctcatgacgctgcggctgtggtccagctgaggtgaggggccttgaagctgggagtggggtttagggacgcgggtctctgggtgcatcctaagctctgagagcaaacctccctgcagggtcttgcttttaagtccaaagcctgagcccaccaaactctcctacttcttcctgttacaaattcctcttgtgcaataataatggcctgaaacgctgtaaaatatcctcatttcagccgcctcagttgcacttctcccctatgaggtaggaagaacagttgtttagaaacgaagaaactgaggccccacagctaatgagtggaggaagagagacacttgtgtacaccacatgccttgtgttgtacttctctcaccgtgtaacctcctcatgtcctctctccccagtacggctctcttagctcagtagaaagaagacattacactcatattacaccccaatcctggctagagtctccgcaccctcctcccccagggtccccagtcgtcttgctgacaactgcatcctgttccatcaccatcaaaaaaaaactccaggctgggtgcgggggctcacacctgtaatcccagcactttgggaggcagaggcaggaggagcacaggagctggagaccagcctgggcaacacagggagaccccgcctctacaaaaagtgaaaaaattaaccaggtgtggtgctgcacacctgtagtcccagctacttaagaggctgagatgggaggatcgcttgagccctggaatgttgaggctacaatgagctgtgattgcgtcactgcactccagcctggaagacaaagcaagatcctgtctcaaataataaaaaaaataagaactccagggtacatttgctcctagaactctaccacatagccccaaacagagccatcaccatcacatccctaacagtcctgggtcttcctcagtgtccagcctgacttctgttcttcctcattccagatctgcaagattgtaagacagcctgtgctccctcgctccttcctctgcattgcccctcttctccctctccaaacagagggaactctcctacccccaaggaggtgaaagctgctaccacctctgtgcccccccggcaatgccaccaactggatcctacccgaatttatgattaagattgctgaagagctgccaaacactgctgccaccccctctgttcccttattgctgcttgtcactgcctgacattcacggcagaggcaaggctgctgcagcctcccctggctgtgcacattccctcctgctccccagagactgcctccgccatcccacagatgatggatcttcagtgggttctcttgggctctaggtcctgcagaatgttgtgaggggtttatttttttttaatagtgttcataaagaaatacatagtattcttcttctcaagacgtggggggaaattatctcattatcgaggccctgctatgctgtgtatctgggcgtgttgtatgtcctgctgccgatgccttc

SEQ ID NO: 4 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - DNA, 22 bp, Homo sapiens
  source 1..22: mol_type = genomic DNA
tggcctggagcaacaaatctga

SEQ ID NO: 6 - DNA, 22 bp, Homo sapiens
  source 1..22: mol_type = genomic DNA
accggacctcgttgtttagact

SEQ ID NO: 7 - protein, 354 aa, synthetic construct
MNTKYNKEFLLYLAGFVDADGSIYAVIYPHQRAKFKYFLKLLFTVNQSTKRRWFLDKLVDEIGVGYVYDGPRTSEYHLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQCSKFKHRLTLGFAVGQKTQRRWFLDKLVDEIGVGYVYDRGSVSEYVLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 8 - protein, 354 aa, synthetic construct
MNTKYNKEFLLYLAGFVDADGSIYAVIYPHQRAKFKHFLKLLFTVNQSTKRRWFLDKLVDEIGVGYVYDGPRTSEYHLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQCSKFKHRLTLGFAVGQKTQRRWFLDKLVDEIGVGYVYDRGSVSEYVLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 9 - protein, 354 aa, synthetic construct
MNTKYNKEFLLYLAGFVDADGSIYAVIYPHQRAKFKHFLKLLFTVNQSTKRRWFLDKLVDEIGVGYVYDGPRTSEYHLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQCSKFKHRLTLGFAVGQKTQRRWFLDKLVDEIGVGYVYDRGSVSEYVLSQIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 10 - protein, 354 aa, synthetic construct
MNTKYNKEFLLYLAGFVDADGSIYAVIYPHQRAKFKHFLKLLFTVNQSTKRRWFLDKLADEIGVGYVYDGPRTSEYHLSQIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQCSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 11 - protein, 147 aa, synthetic construct
KEFLLYLAGFVDGDGSIYACIRPRQCSKFKHRLTLGFAVGQKTQRRWFLDKLVDEIGVGYVYDRGSVSEYVLSQIKPLHNFLTQLQPFLKLKQKQANLVLKIIEQLPSAKESPDKFLEVCTWVDQIAALNDSKTRKTTSETVRAVLD

SEQ ID NO: 12 - protein, 147 aa, synthetic construct; identical to SEQ ID NO: 11

SEQ ID NO: 13 - protein, 147 aa, synthetic construct; identical to SEQ ID NO: 11

SEQ ID NO: 14 - protein, 147 aa, synthetic construct
KEFLLYLAGFVDGDGSIYACIRPRQCSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLD

SEQ ID NO: 15 - protein, 147 aa, synthetic construct
KEFLLYLAGFVDADGSIYAVIYPHQRAKFKYFLKLLFTVNQSTKRRWFLDKLVDEIGVGYVYDGPRTSEYHLSEIKPLHNFLTQLQPFLKLKQKQANLVLKIIEQLPSAKESPDKFLEVCTWVDQIAALNDSRTRKTTSETVRAVLD

SEQ ID NO: 16 - protein, 147 aa, synthetic construct
KEFLLYLAGFVDADGSIYAVIYPHQRAKFKHFLKLLFTVNQSTKRRWFLDKLVDEIGVGYVYDGPRTSEYHLSEIKPLHNFLTQLQPFLKLKQKQANLVLKIIEQLPSAKESPDKFLEVCTWVDQIAALNDSRTRKTTSETVRAVLD

SEQ ID NO: 17 - protein, 147 aa, synthetic construct
KEFLLYLAGFVDADGSIYAVIYPHQRAKFKHFLKLLFTVNQSTKRRWFLDKLVDEIGVGYVYDGPRTSEYHLSQIKPLHNFLTQLQPFLKLKQKQANLVLKIIEQLPSAKESPDKFLEVCTWVDQIAALNDSRTRKTTSETVRAVLD

SEQ ID NO: 18 - protein, 147 aa, synthetic construct
KEFLLYLAGFVDADGSIYAVIYPHQRAKFKHFLKLLFTVNQSTKRRWFLDKLADEIGVGYVYDGPRTSEYHLSQIKPLHNFLTQLQPFLKLKQKQANLVLKIIEQLPSAKESPDKFLEVCTWVDQIAALNDSRTRKTTSETVRAVLD

SEQ ID NO: 19 - DNA, 1062 bp, synthetic construct
  source 1..1062: mol_type = other DNA
atgaatacaaaatataataaagagttcttactctacttagcagggtttgtagacgctgacggttccatctatgctgttatctatcctcatcaacgtgctaagttcaagtacttcctgaagctgcttttcacggtcaatcagagtacaaagcgccgttggttcctcgacaagctggtggacgagatcggtgtgggttacgtgtatgacgggccgcgtacgtccgagtaccatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaggacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcctgtatccggccgaggcagtgtagtaagttcaagcacaggctgactctggggttcgcggtcgggcagaagacacagcgccgttggttcctcgacaagctggtggacgagatcggtgtgggttacgtgtatgacagaggcagcgtctccgagtacgtgctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 20 - DNA, 1062 bp, synthetic construct
  source 1..1062: mol_type = other DNA
atgaatacaaaatataataaagagttcttactctacttagcagggtttgtagacgctgacggttccatctatgctgttatctatcctcatcaacgtgctaagttcaagcacttcctgaagctgcttttcacggtcaatcagagtacaaagcgccgttggttcctcgacaagctggtggacgagatcggtgtgggttacgtgtatgacgggccgcgtacgtccgagtaccatctgtccgagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaggacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcctgtatccggccgaggcagtgtagtaagttcaagcacaggctgactctggggttcgctgtcgggcagaagacacagcgccgttggttcctcgacaagctggtggacgagatcggtgtgggttacgtgtatgacagaggcagcgtctccgagtacgtgctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 21 - DNA, 1062 bp, synthetic construct
  source 1..1062: mol_type = other DNA
atgaatacaaaatataataaagagttcttactctacttagcagggtttgtagacgctgacggttccatctatgctgttatctatcctcatcaacgtgcgaagttcaagcacttcctgaagctgcttttcacggtcaatcagagtacaaagcgccgttggttcctcgacaagctggtggacgagatcggtgtgggttacgtgtatgacgggccgcgtacgtccgagtaccatctgtcccagatcaagcctttgcataattttttaacacaactacaaccttttctaaaactaaaacaaaaacaagcaaatttagttttaaaaattattgaacaacttccgtcagcaaaagaatccccggacaaattcttagaagtttgtacatgggtggatcaaattgcagctctgaatgattcgaggacgcgtaaaacaacttctgaaaccgttcgtgctgtgctagacagtttaccaggatccgtgggaggtctatcgccatctcaggcatccagcgccgcatcctcggcttcctcaagcccgggttcagggatctccgaagcactcagagctggagcaggttccggcactggatacaacaaggaattcctgctctacctggcgggcttcgtcgacggggacggctccatctatgcctgtatccggccgaggcagtgtagtaagttcaagcacaggctgactctggggttcgctgtcgggcagaagacacagcgccgttggttcctcgacaagctggtggacgagatcggtgtgggttacgtgtatgacagaggcagcgtctccgagtacgtgctgtcccagatcaagcctctgcacaacttcctgacccagctccagcccttcctgaagctcaagcagaagcaggccaacctcgtgctgaagatcatcgagcagctgccctccgccaaggaatccccggacaagttcctggaggtgtgcacctgggtggaccagatcgccgctctgaacgactccaagacccgcaagaccacttccgaaaccgtccgcgccgttctagacagtctctccgagaagaagaagtcgtccccc

SEQ ID NO: 22 - DNA, 1062 bp, synthetic construct; identical to SEQ ID NO: 20
  source 1..1062: mol_type = other DNA

SEQ ID NO: 23 - protein, 354 aa, synthetic construct
MNTKYNKEFLLYLAGFVDGDGSIFASIYPHQRAKFKHFLKLTFAVYQKTQRRWFLDKLVDEIGVGYVYDSGSVSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIAPRQGSKFKHRLKLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 24 - protein, 354 aa, synthetic construct
MNTKYNKEFLLYLAGFVDGDGSIYAVIYPHQRAKFKHFLKLLFTVSQSTKRRWFLDKLVDEIGVGYVYDLPRTSEYRLSEIKPLHNFLTQLQPFLKLKQKQANLVLKIIEQLPSAKESPDKFLEVCTWVDQIAALNDSRTRKTTSETVRAVLDSLPGSVGGLSPSQASSAASSASSSPGSGISEALRAGAGSGTGYNKEFLLYLAGFVDGDGSIYACIRPRQGSKFKHRLTLGFAVGQKTQRRWFLDKLVDEIGVGYVYDRGSVSEYVLSEIKPLHNFLTQLQPFLKLKQKQANLVLKIIEQLPSAKESPDKFLEVCTWVDQIAALNDSKTRKTTSETVRAVLDSLSEKKKSSP

SEQ ID NO: 25 - protein, 10 aa, synthetic construct
MAPKKKRKVH

SEQ ID NO: 26 - protein, 9 aa, synthetic construct
PAAKRVKLD

(19) United States
(12) Patent Application Publication
(10) Pub. No.: US 2026/0117212 A1
(43) Pub. Date: Apr. 30, 2026

(54) OPTIMIZED ENGINEERED MEGANUCLEASES HAVING SPECIFICITY FOR THE HUMAN T CELL RECEPTOR ALPHA CONSTANT REGION GENE

(71) Applicant: Precision BioSciences, Inc., Durham, NC (US)

(72) Inventors: James Jefferson Smith, Morrisville, NC (US); Caitlin Turner, Durham, NC (US)

(73) Assignee: Precision BioSciences, Inc., Durham, NC (US)

(21) Appl. No.: 19/145,413

(22) PCT Filed: Jan. 4, 2024
(86) PCT No.: PCT/US2024/010324

§ 371 Date: Jul. 2, 2025

Related U.S. Application Data
(60) Provisional application No. 63/478,592, filed on Jan. 5, 2023.

(51) Int. Cl. C12N 9/22

(52) U.S. Cl. CPC C12N 9/22

(57) ABSTRACT

The present invention encompasses engineered meganucleases that bind and cleave a recognition sequence within the first exon of the human T cell receptor (TCR) alpha constant region gene. The engineered meganucleases can exhibit at least one optimized characteristic, such as increased specificity or efficiency of cleavage, when compared to previous generations of meganucleases. The present invention also encompasses methods of using such engineered nucleases to make genetically-modified cells, and the use of such cells in a pharmaceutical composition and in methods for treating diseases, such as cancer.

### FIELD OF THE INVENTION

The invention relates to the field of oncology, cancer immunotherapy, molecular biology and recombinant nucleic acid technology. In particular, the invention relates to optimized engineered meganucleases having specificity for a recognition sequence in the human T cell receptor alpha constant region gene. The invention further relates to the use of such engineered meganucleases in methods for producing genetically-modified T cells as well as methods of using such cells for treating a disease, including cancer, in a subject.

### REFERENCE TO A SEQUENCE LISTING SUBMITTED AS A TEXT FILE VIA EFS-WEB

The instant application contains a Sequence Listing which has been submitted in XML format via USPTO Patent Center and is hereby incorporated by reference in its entirety. Said XML copy, created on Jan. 4, 2023 is named P89339_1240US_P1_Seq_List_xml, and is 34.9 KB in size.

### BACKGROUND OF THE INVENTION

T cell adoptive immunotherapy is a promising approach for cancer treatment. This strategy utilizes isolated human T cells that have been genetically-modified to enhance their specificity for a specific tumor associated antigen. Genetic modification may involve the expression of a chimeric antigen receptor or an exogenous T cell receptor to graft antigen specificity onto the T cell. By contrast to exogenous T cell receptors, chimeric antigen receptors derive their specificity from the variable domains of a monoclonal antibody. Thus, T cells expressing chimeric antigen receptors (CAR T cells) induce tumor immunoreactivity in a major histocompatibility complex non-restricted manner. T cell adoptive immunotherapy has been utilized as a clinical therapy for a number of cancers, including B cell malignancies (e.g., acute lymphoblastic leukemia, B cell non-Hodgkin lymphoma, acute myeloid leukemia, and chronic lymphocytic leukemia), multiple myeloma, neuroblastoma, glioblastoma, advanced gliomas, ovarian cancer, mesothelioma, melanoma, prostate cancer, pancreatic cancer, and others.

Despite its potential usefulness as a cancer treatment, adoptive immunotherapy with CAR T cells has been limited, in part, by expression of the endogenous T cell receptor on the cell surface. CAR T cells expressing an endogenous T cell receptor may recognize major and minor histocompatibility antigens following administration to an allogeneic patient, which can lead to the development of graft-versus-host-disease (GVHD). As a result, clinical trials have largely focused on the use of autologous CAR T cells, wherein a patient's T cells are isolated, genetically-modified to incorporate a chimeric antigen receptor, and then re-infused into the same patient. An autologous approach provides immune tolerance to the administered CAR T cells; however, this approach is constrained by both the time and expense necessary to produce patient-specific CAR T cells after a patient's cancer has been diagnosed.

Thus, many recent efforts have been made to develop “off the shelf” CAR T cells, prepared using T cells from a third party, healthy donor, that have reduced expression of the endogenous T cell receptor and do not initiate GVHD upon administration. Such products could be generated and validated in advance of diagnosis, and could be made available to patients as soon as necessary. Therefore, a need exists for the development of allogeneic CAR T cells that lack an endogenous T cell receptor in order to prevent the occurrence of GVHD.

Genetic modification of genomic DNA can be performed using site-specific, rare-cutting endonucleases that are engineered to recognize DNA sequences in the locus of interest. Homing endonucleases are a group of naturally-occurring nucleases that recognize 15-40 base-pair cleavage sites commonly found in the genomes of plants and fungi. They are frequently associated with parasitic DNA elements, such as group 1 self-splicing introns and inteins. They naturally promote homologous recombination or gene insertion at specific locations in the host genome by producing a double-stranded break in the chromosome, which recruits the cellular DNA-repair machinery (Stoddard (2006), Q. Rev. Biophys. 38:49-95). Homing endonucleases are commonly grouped into four families: the LAGLIDADG (SEQ ID NO: 2) family, the GIY-YIG family, the His-Cys box family and the HNH family. These families are characterized by structural motifs, which affect catalytic activity and recognition sequence. For instance, members of the LAGLIDADG (SEQ ID NO: 2) family are characterized by having either one or two copies of the conserved LAGLIDADG (SEQ ID NO: 2) motif (see Chevalier et al. (2001), Nucleic Acids Res. 29 (18): 3757-3774). The LAGLIDADG (SEQ ID NO: 2) homing endonucleases with a single copy of the LAGLIDADG (SEQ ID NO: 2) motif form homodimers, whereas members with two copies of the LAGLIDADG (SEQ ID NO: 2) motif are found as monomers.

I-CreI (SEQ ID NO: 1) is a member of the LAGLIDADG (SEQ ID NO: 2) family of homing endonucleases that recognizes and cuts a 22 basepair recognition sequence in the chloroplast chromosome of the algae Chlamydomonas reinhardtii. Genetic selection techniques have been used to modify the wild-type I-CreI cleavage site preference (Sussman et al. (2004), J. Mol. Biol. 342:31-41; Chames et al. (2005), Nucleic Acids Res. 33: e178; Seligman et al. (2002), Nucleic Acids Res. 30:3870-9, Arnould et al. (2006), J. Mol. Biol. 355:443-58). More recently, a method of rationally-designing mono-LAGLIDADG (SEQ ID NO: 2) homing endonucleases was described that is capable of comprehensively redesigning I-CreI and other homing endonucleases to target widely-divergent DNA sites, including sites in mammalian, yeast, plant, bacterial, and viral genomes (WO 2007/047859).

As first described in WO 2009/059195, I-CreI and its engineered derivatives are normally dimeric but can be fused into a single polypeptide using a short peptide linker that joins the C-terminus of a first subunit to the N-terminus of a second subunit (Li et al. (2009), Nucleic Acids Res. 37:1650-62; Grizot et al. (2009), Nucleic Acids Res. 37:5405-19). Thus, a functional “single-chain” meganuclease can be expressed from a single transcript.

The use of nucleases for disrupting expression of the endogenous TCR has been disclosed, including the use of small-hairpin RNAs, zinc finger nucleases (ZFNs), transcription activator-like effector nucleases (TALENs), megaTALs, and CRISPR systems (e.g., Osborn et al. (2016), Molecular Therapy 24 (3): 570-581; Eyquem et al. (2017), Nature 543:113-117; U.S. Pat. No. 8,956,828; U.S. Publication No. US2014/0301990; U.S. Publication No. US2012/0321667).

The specific use of engineered meganucleases for cleaving DNA targets in the human TCR alpha constant region gene has also been previously disclosed. For example, International Publication No. WO 2014/191527 disclosed variants of the I-OnuI meganuclease that were also engineered to target a recognition sequence (SEQ ID NO: 3 of the '527 publication) within exon 1 of the TCR alpha constant region gene. Although the '527 publication discusses that a chimeric antigen receptor can be expressed in TCR knockout cells, the authors did not disclose the insertion of the CAR coding sequence into the meganuclease cleavage site.

Moreover, in International Publication Nos. WO 2017/062439 and WO 2017/062451, Applicants disclosed engineered meganucleases which have specificity for recognition sequences in exon 1 of the TCR alpha constant region gene. These included “TRC 1-2 meganucleases” which have specificity for the TRC 1-2 recognition sequence (SEQ ID NO: 5) in exon 1. The '439 and '451 publications also disclosed methods for targeted insertion of a CAR coding sequence or an exogenous TCR coding sequence into the TCR 1-2 meganuclease cleavage site. Additionally, Applicants further disclosed in International Publication No. WO 2019/200122 several second-generation TRC 1-2 meganuclease with improved characteristics.

In the present invention, Applicants have further improved upon the nucleases and methods taught in the prior art. Through extensive experimentation, Applicants have generated novel, third-generation TRC 1-2 meganucleases which comprise unique, unpredictable combinations of residues and are unexpectedly superior to the first-generation and second-generation TRC 1-2 meganucleases taught in the '439, '451, and '122 applications.

### SUMMARY OF THE INVENTION

In one aspect, the invention provides an engineered meganuclease that binds and cleaves a recognition sequence comprising SEQ ID NO: 5 in a T cell receptor alpha constant region (TRAC) gene, wherein the engineered meganuclease comprises a first subunit and a second subunit, wherein the first subunit binds to a first recognition half-site of the recognition sequence and comprises a first hypervariable (HVR1) region, and wherein the second subunit binds to a second recognition half-site of the recognition sequence and comprises a second hypervariable (HVR2) region.

In some embodiments, the HVR1 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 215-270 of any one of SEQ ID NOs: 7-10.

In some embodiments, the HVR1 region comprises one or more residues corresponding to residues 215, 217, 219, 221, 223, 224, 229, 231, 233, 235, 237, 259, 261, 266, and 268 of any one of SEQ ID NOs: 7-10.

In some embodiments, the HVR1 region comprises residues corresponding to residues 215, 217, 219, 221, 223, 224, 229, 231, 233, 235, 237, 259, 261, 266, and 268 of any one of SEQ ID NOs: 7-10.

In some embodiments, the HVR1 region comprises Y, R, K, or D at a residue corresponding to residue 257 of any one of SEQ ID NOs: 7-10.

In some embodiments, the HVR1 region comprises residues 215-270 of any one of SEQ ID NOs: 7-10.

In some embodiments, the first subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 198-344 of any one of SEQ ID NOs: 7-10.

In some embodiments, the first subunit comprises a residue corresponding to residue 271 of any one of SEQ ID NOs: 7-9.

In some embodiments, the first subunit comprises G, S, or A at a residue corresponding to residue 210 of any one of SEQ ID NOs: 7-10.

In some embodiments, the first subunit comprises E, Q, or K at a residue corresponding to residue 271 of any one of SEQ ID NOs: 7-10.

In some embodiments, the first subunit comprises residues 198-344 of any one of SEQ ID NOs: 7-10.

In some embodiments, the HVR2 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 24-79 of any one of SEQ ID NOs: 7-10.

In some embodiments, the HVR2 region comprises one or more residues corresponding to residues 24, 26, 28, 30, 32, 33, 38, 40, 42, 44, 46, 68, 70, 75, and 77 of any one of SEQ ID NOs: 7-10.

In some embodiments, the HVR2 region comprises residues corresponding to residues 24, 26, 28, 30, 32, 33, 38, 40, 42, 44, 46, 68, 70, 75, and 77 of any one of SEQ ID NOs: 7-10.

In some embodiments, the HVR2 region comprises a residue corresponding to residue 37 of SEQ ID NO: 7.

In some embodiments, the HVR2 region comprises a residue corresponding to residue 48 of any one of SEQ ID NOs: 7-10.

In some embodiments, the HVR2 region comprises a residue corresponding to residue 50 of any one of SEQ ID NOs: 7-10.

In some embodiments, the HVR2 region comprises a residue corresponding to residue 59 of SEQ ID NO: 10.

In some embodiments, the HVR2 region comprises a residue corresponding to residue 71 of any one of SEQ ID NOs: 7-10.

In some embodiments, the HVR2 region comprises a residue corresponding to residue 72 of any one of SEQ ID NOs: 7-10.

In some embodiments, the HVR2 region comprises a residue corresponding to residue 73 of any one of SEQ ID NOs: 7-10.

In some embodiments, the HVR2 region comprises Y, R, K, or D at a residue corresponding to residue 257 of any one of SEQ ID NOs: 7-10.

In some embodiments, the HVR2 region comprises residues 24-79 of any one of SEQ ID NOs: 7-10.

In some embodiments, the second subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 7-153 of any one of SEQ ID NOs: 7-10.

In some embodiments, the second subunit comprises a residue corresponding to residue 19 of any one of SEQ ID NOs: 7-10.

In some embodiments, the second subunit comprises a residue corresponding to residue 80 of SEQ ID NO: 9 or 10.

In some embodiments, the second subunit comprises a residue corresponding to residue 139 of any one of SEQ ID NOs: 7-10.

In some embodiments, the second subunit comprises G, S, or A at a residue corresponding to residue 19 of any one of SEQ ID NOs: 7-10.

In some embodiments, the second subunit comprises E, Q, or K at a residue corresponding to residue 80 of any one of SEQ ID NOs: 7-10.

In some embodiments, the second subunit comprises residues 7-153 of any one of SEQ ID NOs: 7-10.

In some embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to any one of SEQ ID NOs: 7-10.

In some embodiments, the engineered meganuclease comprises an amino acid sequence having of any one of SEQ ID NOs: 7-10.

In some embodiments, the engineered meganuclease is encoded by a nucleic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to a nucleic acid sequence set forth in any one of SEQ ID NOs: 19-22.

In some embodiments, the engineered meganuclease is encoded by a nucleic acid sequence set forth in any one of SEQ ID NOs: 19-22.

In some embodiments, the engineered meganuclease exhibits at least one of the following optimized characteristics as compared to the TRC 1-2L.1592 meganuclease set forth in SEQ ID NO: 24: improved on-target specificity, enhanced generation of insertions or deletions at the recognition sequence, and enhanced transgene insertion at the recognition sequence.

In each of the embodiments above, the engineered meganuclease can comprise a nuclear localization signal (NLS). In some embodiments, the NLS is at the N-terminus of the engineered meganuclease. In some embodiments, the NLS is at the C-terminus of the engineered meganuclease. In some embodiments, the engineered meganuclease comprises a first NLs at the N-terminus and a second NLS at the C-terminus. In some embodiments, the first and second NLS are identical. In some embodiments, the first and second NLS are not identical. In some embodiments, engineered meganuclease comprises an NLS comprising an amino acid sequence having at least 80% or at least 90% sequence identity to a sequence set forth in SEQ ID NO: 25. In some embodiments, the NLS comprises a sequence set forth in SEQ ID NO: 25. In some embodiments, engineered meganuclease comprises an NLS comprising an amino acid sequence having at least 80% or at least 90% sequence identity to a sequence set forth in SEQ ID NO: 26. In some embodiments, the NLS comprises a sequence set forth in SEQ ID NO: 26.

In another aspect, the invention provides a polynucleotide comprising a nucleic acid sequence encoding an engineered meganuclease described herein.

In some embodiments, the polynucleotide is an mRNA.

In another aspect, the invention provides a recombinant DNA construct comprising a polynucleotide comprising a nucleic acid sequence encoding an engineered meganuclease described herein.

In some embodiments, the recombinant DNA construct encodes a recombinant virus comprising the polynucleotide.

In some embodiments, the recombinant virus is a recombinant adenovirus, a recombinant lentivirus, a recombinant retrovirus, or a recombinant adeno-associated virus (AAV).

In some embodiments, the recombinant virus is a recombinant AAV.

In some embodiments, the recombinant AAV has a serotype of AAV6.

In another aspect, the invention provides a recombinant virus comprising a polynucleotide comprising a nucleic acid sequence encoding an engineered meganuclease described herein.

In some embodiments, the recombinant virus is a recombinant adenovirus, a recombinant lentivirus, a recombinant retrovirus, or a recombinant AAV.

In some embodiments, the recombinant virus is a recombinant AAV.

In some embodiments, the recombinant AAV has a serotype of AAV6.

In another aspect, the invention provides a lipid nanoparticle composition comprising lipid nanoparticles comprising a polynucleotide, wherein the polynucleotide comprises a nucleic acid sequence encoding an engineered meganuclease described herein.

In some embodiments, the polynucleotide is an mRNA.

In another aspect, the invention provides a method for producing a genetically-modified eukaryotic cell comprising an exogenous sequence of interest inserted into a chromosome of the eukaryotic cell, the method comprising introducing into a eukaryotic cell one or more nucleic acids including: (a) a first nucleic acid encoding an engineered meganuclease described herein, wherein the engineered meganuclease is expressed in the eukaryotic cell; and (b) a second nucleic acid including the sequence of interest; wherein the engineered meganuclease produces a cleavage site in the chromosome at a recognition sequence comprising SEQ ID NO: 5; and wherein the sequence of interest is inserted into the chromosome at the cleavage site.

In some embodiments of the method, the second nucleic acid further comprises sequences homologous to sequences flanking the cleavage site, and the sequence of interest is inserted at the cleavage site by homologous recombination.

In some embodiments of the method, the genetically-modified eukaryotic cell does not exhibit cell surface expression of an endogenous alpha/beta T cell receptor.

In some embodiments of the method, the eukaryotic cell is a human T cell, or a cell derived therefrom. In some embodiments of the method, the eukaryotic cell is a human NK cell, or a cell derived therefrom. In some embodiments of the method, the eukaryotic cell is a human induced pluripotent stem cell (iPSC), or a cell derived therefrom.

In some embodiments of the method, the sequence of interest comprises a coding sequence for a chimeric antigen receptor (CAR). In some embodiments of the method, the sequence of interest comprises a coding sequence for an exogenous T cell receptor (TCR).

In some embodiments of the method, the CAR or the exogenous TCR comprises an extracellular ligand-binding domain having specificity for a tumor-specific antigen.

In some embodiments of the method, the first nucleic acid is introduced into the eukaryotic cell by an mRNA.

In some embodiments of the method, the mRNA is introduced into the eukaryotic cell by a lipid nanoparticle.

In some embodiments of the method, the mRNA is introduced into the eukaryotic cell by electroporation.

In some embodiments of the method, the second nucleic acid is introduced into the eukaryotic cell by a viral vector comprising the second nucleic acid in its viral genome.

In some embodiments of the method, the viral vector is an adenoviral vector, a lentiviral vector, a retroviral vector, or an AAV vector.

In some embodiments of the method, the viral vector is a recombinant AAV vector.

In some embodiments of the method, the recombinant AAV has a serotype of AAV6.

In another aspect, the invention provides a method for producing a genetically-modified eukaryotic cell comprising an exogenous sequence of interest inserted into a chromosome of the eukaryotic cell, the method comprising: (a) introducing an engineered meganuclease described herein into a eukaryotic cell; and (b) introducing a nucleic acid comprising the sequence of interest into the eukaryotic cell; wherein the engineered meganuclease produces a cleavage site in the chromosome at a recognition sequence comprising SEQ ID NO: 5; and wherein the sequence of interest is inserted into the chromosome at the cleavage site.

In some embodiments of the method, the nucleic acid further comprises sequences homologous to sequences flanking the cleavage site, and the sequence of interest is inserted at the cleavage site by homologous recombination.

In some embodiments of the method, the genetically-modified eukaryotic cell does not exhibit cell surface expression of an endogenous alpha/beta T cell receptor.

In some embodiments of the method, the eukaryotic cell is a human T cell, or a cell derived therefrom. In some embodiments of the method, the eukaryotic cell is a human NK cell, or a cell derived therefrom. In some embodiments of the method, the eukaryotic cell is a human iPSC.

In some embodiments of the method, the sequence of interest comprises a coding sequence for a chimeric antigen receptor (CAR). In some embodiments of the method, the sequence of interest comprises a coding sequence for an exogenous T cell receptor (TCR).

In some embodiments of the method, the CAR or the exogenous TCR comprises an extracellular ligand-binding domain having specificity for a tumor-specific antigen.

In some embodiments of the method, the nucleic acid is introduced into the eukaryotic cell by a viral vector comprising the nucleic acid in its viral genome.

In some embodiments of the method, the viral vector is an adenoviral vector, a lentiviral vector, a retroviral vector, or an AAV vector.

In some embodiments of the method, the viral vector is a recombinant AAV vector.

In some embodiments of the method, the recombinant AAV has a serotype of AAV6.

In another aspect, the invention provides a method for producing a genetically-modified eukaryotic cell by disrupting a target sequence in a chromosome of the eukaryotic cell, the method comprising: introducing into a eukaryotic cell a nucleic acid encoding an engineered meganuclease described herein, wherein the engineered meganuclease is expressed in the eukaryotic cell; wherein the engineered meganuclease produces a cleavage site in the chromosome at a recognition sequence comprising SEQ ID NO: 5, and wherein the target sequence is disrupted by non-homologous end-joining at the cleavage site.

In some embodiments of the method, the genetically-modified eukaryotic cell does not exhibit cell surface expression of an endogenous alpha/beta T cell receptor.

In some embodiments of the method, the eukaryotic cell is a human T cell, or a cell derived therefrom. In some embodiments of the method, the eukaryotic cell is a human NK cell, or a cell derived therefrom. In some embodiments of the method, the eukaryotic cell is a human iPSC.

In some embodiments of the method, the eukaryotic cell expresses a CAR. In some embodiments of the method, the eukaryotic cell expresses an exogenous TCR.

In some embodiments of the method, the nucleic acid is introduced into the eukaryotic cell by an mRNA.

In some embodiments of the method, the mRNA is introduced into the eukaryotic cell by a lipid nanoparticle.

In some embodiments of the method, the mRNA is introduced into the eukaryotic cell by electroporation.

In another aspect, the invention provides a method for producing a genetically-modified eukaryotic cell by disrupting a target sequence in a chromosome of the eukaryotic cell, the method comprising: introducing into a eukaryotic cell an engineered meganuclease described herein; wherein the engineered meganuclease produces a cleavage site in the chromosome at a recognition sequence comprising SEQ ID NO: 5, and wherein the target sequence is disrupted by non-homologous end-joining at the cleavage site.

In some embodiments of the method, the genetically-modified eukaryotic cell does not exhibit cell surface expression of an endogenous alpha/beta T cell receptor.

In some embodiments of the method, the eukaryotic cell is a human T cell, or a cell derived therefrom. In some embodiments of the method, the eukaryotic cell is a human NK cell, or a cell derived therefrom. In some embodiments of the method, the eukaryotic cell is a human iPSC.

In some embodiments of the method, the eukaryotic cell expresses a CAR. In some embodiments of the method, the eukaryotic cell expresses an exogenous TCR.

In another aspect, the invention provides a genetically-modified eukaryotic cell prepared by a method described herein wherein an exogenous sequence of interest is inserted into a chromosome of a eukaryotic cell.

In some embodiments, the genetically-modified eukaryotic cell comprises improved on-target specificity, enhanced generation of insertions or deletions at the recognition sequence, and/or enhanced transgene insertion at the recognition sequence by the engineered meganuclease as compared to the TRC 1-2L.1592 meganuclease set forth in SEQ ID NO: 24.

In some embodiments, the genetically-modified eukaryotic cell is a genetically-modified human T cell, or cell derived therefrom. In some embodiments, the genetically-modified eukaryotic cell is a genetically-modified NK cell, or cell derived therefrom. In some embodiments, the genetically-modified eukaryotic cell is a genetically-modified human iPSC.

In some embodiments, the sequence of interest comprises a coding sequence for a CAR. In some embodiments, the sequence of interest comprises a coding sequence for an exogenous TCR.

In some embodiments, the CAR or the exogenous TCR comprises an extracellular ligand-binding domain having specificity for a tumor-specific antigen.

In some embodiments, the genetically-modified eukaryotic cell does not exhibit cell surface expression of an endogenous alpha/beta T cell receptor.

In another aspect, the invention provides a population of genetically-modified eukaryotic cells described herein wherein an exogenous sequence of interest is inserted into a chromosome of a eukaryotic cell, the population comprising a plurality of such genetically-modified eukaryotic cells.

In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100%, of cells in the population are such genetically-modified eukaryotic cells.

In another aspect, the invention provides a genetically-modified eukaryotic cell prepared by a method described herein wherein a target sequence is disrupted in a chromosome of the eukaryotic cell.

In some embodiments, the genetically-modified eukaryotic cell comprises improved on-target specificity, enhanced generation of insertions or deletions at the recognition sequence, and/or enhanced transgene insertion at the recognition sequence by the engineered meganuclease as compared to the TRC 1-2L.1592 meganuclease set forth in SEQ ID NO: 24.

In some embodiments, the genetically-modified eukaryotic cell is a genetically-modified human T cell, or cell derived therefrom. In some embodiments, the genetically-modified eukaryotic cell is a genetically-modified NK cell, or cell derived therefrom. In some embodiments, the genetically-modified eukaryotic cell is a genetically-modified human iPSC.

In some embodiments, the genetically-modified eukaryotic cell expresses a CAR. In some embodiments, the genetically-modified eukaryotic cell expresses an exogenous TCR.

In some embodiments, the exogenous TCR comprises an extracellular ligand-binding domain having specificity for a tumor-specific antigen.

In some embodiments, the genetically-modified eukaryotic cell does not exhibit cell surface expression of an endogenous alpha/beta T cell receptor.

In another aspect, the invention provides a population of genetically-modified eukaryotic cells wherein a target sequence is disrupted in a chromosome of the eukaryotic cell, the population comprising a plurality of such genetically-modified eukaryotic cells.

In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100%, of cells in the population are such genetically-modified eukaryotic cells.

In another aspect, the invention provides a eukaryotic cell comprising an engineered meganuclease described herein.

In some embodiments, the eukaryotic cell is a human T cell, or cell derived therefrom. In some embodiments, the eukaryotic cell is an NK cell, or cell derived therefrom. In some embodiments, the eukaryotic cell is a human iPSC.

In some embodiments, the eukaryotic cell expresses a CAR. In some embodiments, the eukaryotic cell expresses an exogenous TCR.

In some embodiments, the CAR or the exogenous TCR comprises an extracellular ligand-binding domain having specificity for a tumor-specific antigen.

In another aspect, the invention provides a eukaryotic cell comprising a polynucleotide described herein (i.e., comprising a nucleic acid sequence encoding an engineered meganuclease described herein).

In some embodiments, the eukaryotic cell is a human T cell, or cell derived therefrom. In some embodiments, the eukaryotic cell is an NK cell, or cell derived therefrom. In some embodiments, the eukaryotic cell is a human iPSC.

In some embodiments, the eukaryotic cell expresses a CAR. In some embodiments, the eukaryotic cell expresses an exogenous TCR.

In some embodiments, the CAR or the exogenous TCR comprises an extracellular ligand-binding domain having specificity for a tumor-specific antigen.

In another aspect, the invention provides a eukaryotic cell comprising a recombinant DNA construct described herein.

In some embodiments, the eukaryotic cell is a human T cell, or cell derived therefrom. In some embodiments, the eukaryotic cell is an NK cell, or cell derived therefrom. In some embodiments, the eukaryotic cell is a human iPSC.

In some embodiments, the eukaryotic cell expresses a CAR. In some embodiments, the eukaryotic cell expresses an exogenous TCR.

In some embodiments, the CAR or the exogenous TCR comprises an extracellular ligand-binding domain having specificity for a tumor-specific antigen.

In another aspect, the invention provides a eukaryotic cell comprising a recombinant virus described herein.

In some embodiments, the eukaryotic cell is a human T cell, or cell derived therefrom. In some embodiments, the eukaryotic cell is an NK cell, or cell derived therefrom. In some embodiments, the eukaryotic cell is a human iPSC.

In some embodiments, the eukaryotic cell expresses a CAR. In some embodiments, the eukaryotic cell expresses an exogenous TCR.

In some embodiments, the CAR or the exogenous TCR comprises an extracellular ligand-binding domain having specificity for a tumor-specific antigen.

In another aspect, the invention provides a eukaryotic cell comprising a lipid nanoparticle composition described herein.

In some embodiments, the eukaryotic cell is a human T cell, or cell derived therefrom. In some embodiments, the eukaryotic cell is an NK cell, or cell derived therefrom. In some embodiments, the eukaryotic cell is a human iPSC.

In some embodiments, the eukaryotic cell expresses a CAR. In some embodiments, the eukaryotic cell expresses an exogenous TCR.

In some embodiments, the CAR or the exogenous TCR comprises an extracellular ligand-binding domain having specificity for a tumor-specific antigen.

In another aspect, the invention provides a population of eukaryotic cells, the population comprising a plurality of a eukaryotic cell described herein (i.e., comprising an engineered meganuclease described herein, comprising a polynucleotide described herein, comprising a recombinant DNA construct described herein, comprising a recombinant virus described herein, or comprising a lipid nanoparticle composition described herein).

In some embodiments, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or up to 100%, of cells in the population are such eukaryotic cells.

In another aspect, the invention provides a pharmaceutical composition comprising a pharmaceutically-acceptable carrier and a genetically-modified eukaryotic cell described herein wherein an exogenous sequence of interest is inserted into a chromosome of the eukaryotic cell, or a population of genetically-modified eukaryotic cells described herein wherein an exogenous sequence of interest is inserted into a chromosome of the eukaryotic cell.

In some embodiments, the genetically-modified eukaryotic cell or the population is comprised of a genetically-modified human T cell, or a cell derived therefrom. In some embodiments, the genetically-modified eukaryotic cell or the population is comprised of a genetically-modified NK cell, or a cell derived therefrom. In some embodiments, the genetically-modified eukaryotic cell is a genetically-modified human iPSC.

In some embodiments, the sequence of interest comprises a coding sequence for a CAR. In some embodiments, the sequence of interest comprises a coding sequence for an exogenous TCR.

In some embodiments, the CAR or the exogenous TCR comprises an extracellular ligand-binding domain having specificity for a tumor-specific antigen.

In some embodiments, the genetically-modified eukaryotic cell does not exhibit cell surface expression of an endogenous alpha/beta T cell receptor.

In another aspect, the invention provides a pharmaceutical composition comprising a pharmaceutically-acceptable carrier and a genetically-modified eukaryotic cell described herein wherein a target sequence is disrupted in a chromosome of the eukaryotic cell, or a population of genetically-modified eukaryotic cells described herein wherein a target sequence is disrupted in a chromosome of the eukaryotic cell.

In some embodiments, the genetically-modified eukaryotic cell or the population is comprised of a genetically-modified human T cell, or a cell derived therefrom. In some embodiments, the genetically-modified eukaryotic cell or the population is comprised of a genetically-modified NK cell, or a cell derived therefrom.

In some embodiments, the genetically-modified eukaryotic cell expresses a CAR. In some embodiments, the genetically-modified eukaryotic cell expresses an exogenous TCR.

In some embodiments, the CAR or the exogenous TCR comprises an extracellular ligand-binding domain having specificity for a tumor-specific antigen.

In some embodiments, the genetically-modified eukaryotic cell does not exhibit cell surface expression of an endogenous alpha/beta T cell receptor.

In another aspect, the invention provides a method of treating a disease in a subject in need thereof, the method comprising administering to the subject a therapeutically-effective amount of: (a) a genetically-modified eukaryotic cell described herein, or a eukaryotic cell described herein; or (b) a population of genetically-modified eukaryotic cells described herein, or a population of eukaryotic cells described herein.

In some embodiments of the method, the method comprises administering to the subject a pharmaceutical composition described herein.

In some embodiments of the method, the method is an immunotherapy for the treatment of a cancer in a subject in need thereof, wherein the genetically-modified eukaryotic cell is a genetically-modified human T cell, or a cell derived therefrom, or a genetically-modified NK cell, or a cell derived therefrom, and wherein the genetically-modified eukaryotic cell comprises a cell surface CAR or exogenous TCR comprising an extracellular ligand-binding domain having specificity for a tumor-specific antigen, and wherein the genetically-modified eukaryotic cell does not express an endogenous alpha/beta T cell receptor on its cell surface.

In some embodiments of the method, the cancer is selected from the group consisting of a cancer of carcinoma, lymphoma, sarcoma, blastomas, and leukemia.

In some embodiments of the methods, the cancer is selected from the group consisting of a cancer of B-cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin's lymphoma.

In some embodiments of the method, the cancer of B-cell origin is selected from the group consisting of B-lineage acute lymphoblastic leukemia, B-cell chronic lymphocytic leukemia, B-cell non-Hodgkin's lymphoma, and multiple myeloma.

In another aspect, the invention provides a genetically-modified eukaryotic cell or a population thereof, as described herein, for use as a medicament. The invention further provides the use of a genetically-modified eukaryotic cell or a population thereof, as described herein, in the manufacture of a medicament for treating a disease in a subject in need thereof. In one such aspect, the medicament is useful in the treatment of a cancer.

In another aspect, the invention provides a genetically-modified eukaryotic cell or population thereof, as described herein, for use in treatment of a disease, and preferably in the treatment of a cancer.

### BRIEF DESCRIPTION OF THE FIGURES

FIG. 1. TRC 1-2 recognition sequence in the human T cell receptor alpha constant region gene. The TRC 1-2 recognition sequence, targeted by engineered meganucleases of the invention, comprises two recognition half-sites. Each recognition half-site comprises 9 base pairs, separated by a 4 base pair central sequence. The TRC 1-2 recognition sequence (SEQ ID NO: 5) comprises two recognition half-sites referred to as TRC1 and TRC2.

FIG. 2. The engineered meganucleases of the invention comprise two subunits, wherein the first subunit comprising the HVR1 region binds to a first recognition half-site (e.g., TRC1) and the second subunit comprising the HVR2 region binds to a second recognition half-site (e.g., TRC2). In embodiments where the engineered meganuclease is a single-chain meganuclease, the first subunit comprising the HVR1 region can be positioned as either the N-terminal or C-terminal subunit. Likewise, the second subunit comprising the HVR2 region can be positioned as either the N-terminal or C-terminal subunit.

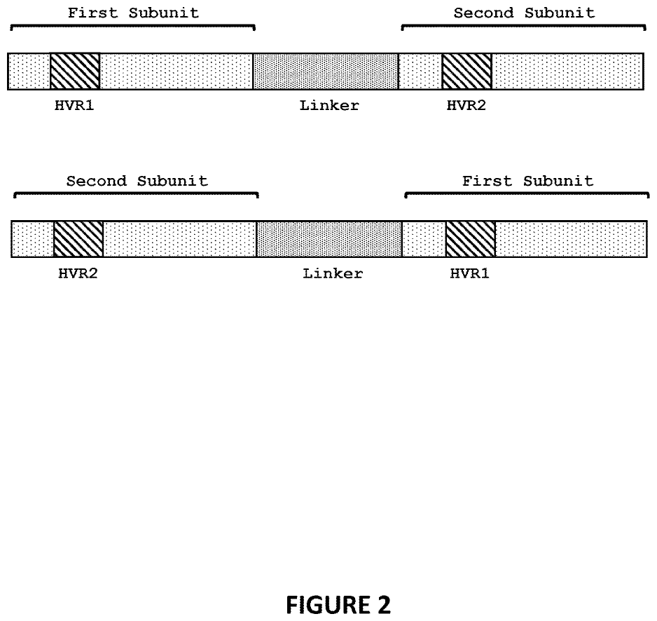

FIG. 3. Alignment of the TRC 1-2L.2307, TRC 1-2L.2213, TRC 1-2L.2231, and TRC 1-2L.2338 engineered meganucleases.

FIG. 4. Graphical visualization of oligo capture data generated for the TRC 1-2x.87 EE, TRC 1-2L.1592, TRC 1-2L.2307, TRC 1-2L.2213, TRC 1-2L.2231, and TRC 1-2L.2338 engineered meganucleases, wherein off target sites are plotted according to their number of aligned reads on the X axis, and the number of mismatched base pairs compared to the intended site are indicated by color, with darker colors indicating closer overall matches between off-targets and the intended binding site. The intended site (i.e., the TRC 1-2 recognition sequence) has the highest read count for each meganuclease tested (circled).

FIG. 5. CAR T cells were produced utilizing mRNA encoding the TRC 1-2x.87 EE, TRC 1-2L.1592, TRC 1-2L.2307, TRC 1-2L.2213, TRC 1-2L.2231, or TRC 1-2L.2338 engineered meganucleases in combination with AAV transduction to provide a donor template encoding a chimeric antigen receptor. The number of CAR T cells present were determined 5 days after electroporation.

FIG. 6. CAR T cells were produced utilizing mRNA encoding the TRC 1-2x.87 EE, TRC 1-2L.1592, TRC 1-2L.2307, TRC 1-2L.2213, TRC 1-2L.2231, or TRC 1-2L.2338 engineered meganucleases in combination with AAV transduction to provide a donor template encoding a chimeric antigen receptor. CAR T cell expansion (FIG. 6A) and cytolysis of target cells (FIG. 6B) were determined after 5 days of co-culture at various effector:target ratios.

FIG. 7. Determination of EC50 and EC90 values in T cells for TRC 1-2L.1592 (FIG. 7A) and TRC 1-2L.2307 (FIG. 7B).

FIG. 8. Characterization of CAR T cell populations generated using TRC 1-2 meganucleases. CAR T cells were produced using either TRC 1-2L.1592 (FIG. 8A) or TRC 1-2L.2307 (FIG. 8B) and characterized by flow cytometry to determine the percentage of cells with knockout of the TRAC locus and/or knock-in of a template encoding a CAR.

FIG. 9. Graph showing the dose response curve of the TRC 1-2L.2307 meganuclease for knocking out cell surface CD3 assessed by flow cytometery. The meganuclease was encoded by the optimized Max construct according to the disclosure herein or by a standard control construct. EC90 and EC50 values are provided for each construct.

### BRIEF DESCRIPTION OF THE SEQUENCES

SEQ ID NO: 1 sets forth the amino acid sequence of the wild-type I-CreI meganuclease from Chlamydomonas reinhardtii.

SEQ ID NO: 2 sets forth the amino acid sequence of the LAGLIDADG motif.

SEQ ID NO: 3 sets forth the nucleic acid sequence of the human T cell receptor alpha constant region gene (NCBI Gene ID NO. 28755).

SEQ ID NO: 4 sets forth the amino acid sequence of the polypeptide encoded by the human T cell receptor alpha constant region gene.

SEQ ID NO: 5 sets forth the nucleic acid sequence of the sense strand of the TRC 1-2 recognition sequence.

SEQ ID NO: 6 sets forth the nucleic acid sequence of the antisense strand of the TRC 1-2 recognition sequence.

SEQ ID NO: 7 sets forth the amino acid sequence of the TRC 1-2L.2307 meganuclease.

SEQ ID NO: 8 sets forth the amino acid sequence of the TRC 1-2L.2213 meganuclease.

SEQ ID NO: 9 sets forth the amino acid sequence of the TRC 1-2L.2231 meganuclease.

SEQ ID NO: 10 sets forth the amino acid sequence of the TRC 1-2L.2338 meganuclease.

SEQ ID NO: 11 sets forth the amino acid sequence of the TRC1 subunit of the TRC 1-2L.2307 meganuclease.

SEQ ID NO: 12 sets forth the amino acid sequence of the TRC1 subunit of the TRC 1-2L.2213 meganuclease.

SEQ ID NO: 13 sets forth the amino acid sequence of the TRC1 subunit of the TRC 1-2L.2231 meganuclease.

SEQ ID NO: 14 sets forth the amino acid sequence of the TRC1 subunit of the TRC 1-2L.2338 meganuclease.

SEQ ID NO: 15 sets forth the amino acid sequence of the TRC2 subunit of the TRC 1-2L.2307 meganuclease.

SEQ ID NO: 16 sets forth the amino acid sequence of the TRC2 subunit of the TRC 1-2L.2213 meganuclease.

SEQ ID NO: 17 sets forth the amino acid sequence of the TRC2 subunit of the TRC 1-2L.2231 meganuclease.

SEQ ID NO: 18 sets forth the amino acid sequence of the TRC2 subunit of the TRC 1-2L.2338 meganuclease.

SEQ ID NO: 19 sets forth the nucleic acid sequence of the TRC 1-2L.2307 meganuclease.

SEQ ID NO: 20 sets forth the nucleic acid sequence of the TRC 1-2L.2213 meganuclease.

SEQ ID NO: 21 sets forth the nucleic acid sequence of the TRC 1-2L.2231 meganuclease.

SEQ ID NO: 22 sets forth the nucleic acid sequence of the TRC 1-2L.2338 meganuclease.

SEQ ID NO: 23 sets forth the amino acid sequence of the TRC 1-2x.87EE meganuclease.

SEQ ID NO: 24 sets forth the amino acid sequence of the TRC 1-2L.1592 meganuclease.

SEQ ID NO: 25 sets forth the amino acid sequence of an SV40 NLS.

SEQ ID NO: 26 sets forth the amino acid sequence of a C-myc NLS.

### DETAILED DESCRIPTION OF THE INVENTION

### 1.1 References and Definitions

The patent and scientific literature referred to herein establishes knowledge that is available to those of skill in the art. The issued US patents, allowed applications, published foreign applications, and references, including GenBank database sequences, which are cited herein are hereby incorporated by reference to the same extent as if each was specifically and individually indicated to be incorporated by reference.

The present invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment can be deleted from that embodiment. In addition, numerous variations and additions to the embodiments suggested herein will be apparent to those skilled in the art in light of the instant disclosure, which do not depart from the instant invention.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference herein in their entirety.

As used herein, “a,” “an,” or “the” can mean one or more than one. For example, “a” cell can mean a single cell or a multiplicity of cells.

As used herein, unless specifically indicated otherwise, the word “or” is used in the inclusive sense of “and/or” and not the exclusive sense of “either/or.”

As used herein, the term “endonuclease” refers to enzymes which cleave a phosphodiester bond within a polynucleotide chain.

As used herein, with respect to double-stranded DNA, the terms “cleave” or “cleavage” refer to the endonuclease-mediated hydrolysis of phosphodiester bonds within the backbone of a recognition sequence within a target sequence that results in a double-stranded break within the target sequence, referred to herein as a “cleavage site”. Depending upon the endonuclease, cleavage can result in double-stranded fragments with blunt ends or fragments with 5′ or 3′ base overhangs.

As used herein, the term “meganuclease” refers to an endonuclease that binds double-stranded DNA at a recognition sequence that is greater than 12 base pairs. In some embodiments, the recognition sequence for a meganuclease of the present disclosure is 22 base pairs. A meganuclease can be an endonuclease that is derived from I-CreI, and can refer to an engineered variant of I-CreI that has been modified relative to natural I-CreI with respect to, for example, DNA-binding specificity, DNA cleavage activity, DNA-binding affinity, or dimerization properties. Methods for producing such modified variants of I-CreI are known in the art (e.g. WO 2007/047859, incorporated by reference in its entirety). A meganuclease as used herein binds to double-stranded DNA as a heterodimer. A meganuclease may also be a “single-chain meganuclease” in which a pair of DNA-binding domains is joined into a single polypeptide using a peptide linker. The term “homing endonuclease” is synonymous with the term “meganuclease.” Meganucleases of the present disclosure are substantially non-toxic when expressed in the targeted cells as described herein, particularly in human T cells, such that cells can be transfected and maintained at 37° C. without observing substantial deleterious effects on overall cell viability or significant reductions in meganuclease cleavage activity when measured using the methods described herein.

As used herein, the term “single-chain meganuclease” refers to a polypeptide comprising a pair of nuclease subunits joined by a linker such that the subunits interact functionally like a heterodimer to cleave a double-stranded recognition site. A single-chain meganuclease has the organization: N-terminal subunit-Linker-C-terminal subunit. The two meganuclease subunits will generally be non-identical in amino acid sequence and will recognize non-identical DNA half-sites within a recognition sequence. Thus, single-chain meganucleases typically cleave pseudo-palindromic or non-palindromic recognition sequences. A single-chain meganuclease may be referred to as a “single-chain heterodimer” or “single-chain heterodimeric meganuclease” although it is not, in fact, dimeric. For clarity, unless otherwise specified, the term “meganuclease” can refer to a dimeric or single-chain meganuclease.

As used herein, the term “linker” refers to an exogenous peptide sequence used to join two meganuclease subunits into a single polypeptide. A linker may have a sequence that is found in natural proteins or may be an artificial sequence that is not found in any natural protein. A linker may be flexible and lacking in secondary structure or may have a propensity to form a specific three-dimensional structure under physiological conditions. A linker can include, without limitation, any of those encompassed by U.S. Pat. Nos. 8,445,251, 9,340,777, 9,434,931, and 10,041,053, each of which is incorporated by reference in its entirety. In some embodiments, a linker may have at least 80%, at least 85%, at least 90%, at least 91%, at least 92%, at least 93%, at least 94%, at least 95%, at least 96%, at least 97%, at least 98%, at least 99%, or more, sequence identity to residues 154-195 of any one of SEQ ID NOs: 7-10. In some embodiments, a linker may have an amino acid sequence comprising residues 154-195 of any one of SEQ ID NOs: 7-10.

As used herein, with respect to a protein, the term “recombinant” or “engineered” means having an altered amino acid sequence as a result of the application of genetic engineering techniques to nucleic acids which encode the protein, and cells or organisms which express the protein. With respect to a nucleic acid, the term “recombinant” or “engineered” means having an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In accordance with this definition, a protein having an amino acid sequence identical to a naturally-occurring protein, but produced by cloning and expression in a heterologous host, is not considered recombinant.

As used herein, the term “wild-type” refers to the most common naturally occurring allele (i.e., polynucleotide sequence) in the allele population of the same type of gene, wherein a polypeptide encoded by the wild-type allele has its original functions. The term “wild-type” also refers to a polypeptide encoded by a wild-type allele. Wild-type alleles (i.e., polynucleotides) and polypeptides are distinguishable from mutant or variant alleles and polypeptides, which comprise one or more mutations and/or substitutions relative to the wild-type sequence(s). Whereas a wild-type allele or polypeptide can confer a normal phenotype in an organism, a mutant or variant allele or polypeptide can, in some instances, confer an altered phenotype. Wild-type nucleases are distinguishable from recombinant or non-naturally-occurring nucleases. The term “wild-type” can also refer to a cell, an organism, and/or a subject which possesses a wild-type allele of a particular gene, or a cell, an organism, and/or a subject used for comparative purposes.

As used herein, the term “genetically-modified” refers to a cell or organism in which, or in an ancestor of which, a genomic DNA sequence has been deliberately modified by recombinant technology. As used herein, the term “genetically-modified” encompasses the term “transgenic.”

As used herein with respect to recombinant proteins, the term “modification” means any insertion, deletion, or substitution of an amino acid residue in the recombinant sequence relative to a reference sequence (e.g., a wild-type or a native sequence).

As used herein, the terms “recognition sequence” or “recognition site” refers to a DNA sequence that is bound and cleaved by an endonuclease. In the case of a meganuclease, a recognition sequence comprises a pair of inverted, 9 basepair “half sites” which are separated by four basepairs. In the case of a single-chain meganuclease, the N-terminal domain of the protein contacts a first half-site and the C-terminal domain of the protein contacts a second half-site. Cleavage by a meganuclease produces four basepair 3′ “overhangs”. “Overhangs,” or “sticky ends” are short, single-stranded DNA segments that can be produced by endonuclease cleavage of a double-stranded DNA sequence. In the case of meganucleases and single-chain meganucleases derived from I-CreI, the overhang comprises bases 10-13 of the 22 basepair recognition sequence.

As used herein, the term “target site” or “target sequence” refers to a region of the chromosomal DNA of a cell comprising a recognition sequence for a nuclease.

As used herein, the term “DNA-binding affinity” or “binding affinity” means the tendency of a meganuclease to non-covalently associate with a reference DNA molecule (e.g., a recognition sequence or an arbitrary sequence). Binding affinity is measured by a dissociation constant, Kd. As used herein, a nuclease has “altered” binding affinity if the Kd of the nuclease for a reference recognition sequence is increased or decreased by a statistically significant percent change, or biologically significant amount (e.g., at least 2×, or 2× to 10×), relative to a reference nuclease.

As used herein, the term “specificity” means the ability of a nuclease to recognize and cleave double-stranded DNA molecules only at a particular sequence of base pairs referred to as the recognition sequence, or only at a particular set of recognition sequences. The set of recognition sequences will share certain conserved positions or sequence motifs, but may be degenerate at one or more positions. A highly-specific nuclease is capable of cleaving only one or a very few recognition sequences. Specificity can be determined by any method known in the art.

As used herein, a nuclease has “altered” specificity if it binds to and cleaves a recognition sequence which is not bound to and cleaved by a reference nuclease (e.g., a wild-type) under physiological conditions, or if the rate of cleavage of a recognition sequence is increased or decreased by a biologically significant amount (e.g., at least 2×, or 2×-10×) relative to a reference nuclease.

In some embodiments, the presently disclosed engineered meganucleases have improved (i.e., increased) specificity for the target recognition sequence of SEQ ID NO: 5 (i.e., TRC 1-2) as compared to the TRC 1-2L.1592 meganuclease (the amino acid sequence of which is set forth as SEQ ID NO: 24). Thus, in certain embodiments, the presently disclosed engineered meganucleases exhibit reduced off-target cleavage as compared to the TRC 1-2L.1592 meganuclease. Off-target cleavage by a meganuclease can be measured using any method known in the art, including for example, oligo capture analysis as described herein, a T7 endonuclease I (T7E) assay, digital PCR, targeted sequencing of particular off-target sites, exome sequencing, whole genome sequencing, direct in situ breaks labeling enrichment on streptavidin and next-generation sequencing (BLESS), genome-wide, unbiased identification of DSBs enabled by sequencing (GUIDE-seq), and linear amplification-mediated high-throughput genome-wide translocation sequencing (LAM-HTGTS) (see, e.g., Zischewski et al. (2017) Biotechnology Advances 35 (1): 95-104, which is incorporated by reference in its entirety).

As used herein, the term “homologous recombination” or “HR” refers to the natural, cellular process in which a double-stranded DNA-break is repaired using a homologous DNA sequence as the repair template (see, e.g. Cahill et al. (2006), Front. Biosci. 11:1958-1976). The homologous DNA sequence may be an endogenous chromosomal sequence or an exogenous nucleic acid that was delivered to the cell.

As used herein, the term “non-homologous end-joining” or “NHEJ” refers to the natural, cellular process in which a double-stranded DNA-break is repaired by the direct joining of two non-homologous DNA segments (see, e.g. Cahill et al. (2006), Front. Biosci. 11:1958-1976). DNA repair by non-homologous end-joining is error-prone and frequently results in the untemplated addition or deletion of DNA sequences at the site of repair. In some instances, cleavage at a target recognition sequence results in NHEJ at a target recognition site. Nuclease-induced cleavage of a target site in the coding sequence of a gene followed by DNA repair by NHEJ can introduce mutations into the coding sequence, such as frameshift mutations, that disrupt gene function. Thus, engineered nucleases can be used to effectively knock-out a gene in a population of cells. As used herein, “disrupting a target sequence” refers to the introduction of a mutation (e.g., frameshift mutation) that interferes with the gene function and prevents expression and/or function of the polypeptide/expression product encoded thereby.

As used herein, a “homology arm” or “sequences homologous to sequences flanking a meganuclease cleavage site” refer to sequences flanking the 5′ and 3′ ends of a nucleic acid molecule which promote insertion of the nucleic acid molecule into a cleavage site generated by a meganuclease. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

As used herein, the term “chimeric antigen receptor” or “CAR” refers to an engineered receptor that confers or grafts specificity for an antigen onto an immune effector cell (e.g., a human T cell). A chimeric antigen receptor comprises at least an extracellular ligand-binding domain or moiety, a transmembrane domain, and an intracellular domain that comprises one or more signaling domains and/or co-stimulatory domains.

In some embodiments, the extracellular ligand-binding domain or moiety is an antibody, or antibody fragment. In this context, the term “antibody fragment” can refer to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some embodiments, the scFv is attached via a linker sequence. In some embodiments, the scFv is murine, humanized, or fully human.

The extracellular ligand-binding domain of a chimeric antigen receptor can also comprise an autoantigen (see, Payne et al. (2016), Science 353 (6295): 179-184), that can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs), and their use is encompassed by the invention. The extracellular ligand-binding domain of a chimeric antigen receptor can also comprise a naturally-occurring ligand for an antigen of interest, or a fragment of a naturally-occurring ligand which retains the ability to bind the antigen of interest.

The intracellular stimulatory domain can include one or more cytoplasmic signaling domains that transmit an activation signal to the T cell following antigen binding. Such cytoplasmic signaling domains can include, without limitation, a CD3 zeta signaling domain.

The intracellular stimulatory domain can also include one or more intracellular co-stimulatory domains that transmit a proliferative and/or cell-survival signal after ligand binding. Such intracellular co-stimulatory domains can be any of those known in the art and can include, without limitation, those co-stimulatory domains disclosed in WO 2018/067697 including, for example, Novel 6. Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof.

A chimeric antigen receptor further includes additional structural elements, including a transmembrane domain that is attached to the extracellular ligand-binding domain via a hinge or spacer sequence. The transmembrane domain can be derived from any membrane-bound or transmembrane protein. For example, the transmembrane polypeptide can be a subunit of the T-cell receptor (e.g., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. In certain examples, the transmembrane domain is a CD8 alpha domain. Alternatively, the transmembrane domain can be synthetic and can comprise predominantly hydrophobic residues such as leucine and valine.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence or may be an entirely synthetic hinge sequence. In particular examples, a hinge domain can comprise a part of a human CD8 alpha chain, FcyRIIIa receptor or IgGI. In certain examples, the hinge region can be a CD8 alpha domain.

As used herein, the terms “exogenous T cell receptor” or “exogenous TCR” refer to a TCR whose sequence is introduced into the genome of a eukaryotic cell (e.g., an immune cell such as a T cell) that may or may not endogenously express the TCR. Expression of an exogenous TCR on a immune cell can confer specificity for a specific epitope or antigen (e.g., an epitope or antigen preferentially present on the surface of a cancer cell or other disease-causing cell or particle). Such exogenous T cell receptors can comprise alpha and beta chains or, alternatively, may comprise gamma and delta chains. Exogenous TCRs useful in the invention may have specificity to any antigen or epitope of interest. Exogenous TCRs can further encompass TCR fusion constructs, such as TRuCs described in, for example, WO2016187349 and WO2021035170.

As used herein, the term “does not exhibit cell surface expression of an endogenous alpha/beta T cell receptor” refers to a knockout (i.e., a 100% knockdown) of cell-surface expression of an endogenous alpha/beta T cell receptor, resulting from genetic inactivation of the T cell receptor alpha constant region gene using the engineered meganucleases described herein. The alpha constant domain encoded by the T cell receptor alpha constant region gene is necessary for assembly of the endogenous TCR complex on the cell surface. Thus, knocking out the T cell receptor alpha constant region gene using engineered meganucleases described herein, either by introduction of an insertion or deletion at the recognition sequence, or by introduction of a donor template into the recognition sequence (e.g., encoding a CAR or exogenous TCR), results in a knockout of cell-surface T cell receptor expression.

As used herein with respect to both amino acid sequences and nucleic acid sequences, the terms “percent identity,” “sequence identity,” “percentage similarity,” “sequence similarity” and the like refer to a measure of the degree of similarity of two sequences based upon an alignment of the sequences which maximizes similarity between aligned amino acid residues or nucleotides, and which is a function of the number of identical or similar residues or nucleotides, the number of total residues or nucleotides, and the presence and length of gaps in the sequence alignment. A variety of algorithms and computer programs are available for determining sequence similarity using standard parameters. As used herein, sequence similarity is measured using the BLASTp program for amino acid sequences and the BLASTn program for nucleic acid sequences, both of which are available through the National Center for Biotechnology Information (www.ncbi.nlm.nih.gov/), and are described in, for example, Altschul et al. (1990), J. Mol. Biol. 215:403-410; Gish and States (1993), Nature Genet. 3:266-272; Madden et al. (1996), Meth. Enzymol. 266:131-141; Altschul et al. (1997), Nucleic Acids Res. 25:33 89-3402); Zhang et al. (2000), J. Comput. Biol. 7 (1-2): 203-14. As used herein, percent similarity of two amino acid sequences is the score based upon the following parameters for the BLASTp algorithm: word size=3; gap opening penalty=−11; gap extension penalty=−1; and scoring matrix=BLOSUM62. As used herein, percent similarity of two nucleic acid sequences is the score based upon the following parameters for the BLASTn algorithm: word size=11; gap opening penalty=−5; gap extension penalty=−2; match reward=1; and mismatch penalty=−3.

As used herein with respect to modifications of two proteins or amino acid sequences, the term “corresponding to” is used to indicate that a specified modification in the first protein is a substitution of the same amino acid residue as in the modification in the second protein, and that the amino acid position of the modification in the first protein corresponds to or aligns with the amino acid position of the modification in the second protein when the two proteins are subjected to standard sequence alignments (e.g., using the BLASTp program). Thus, the modification of residue “X” to amino acid “A” in the first protein will correspond to the modification of residue “Y” to amino acid “A” in the second protein if residues X and Y correspond to each other in a sequence alignment, and despite the fact that X and Y may be different numbers.

As used herein, the term “recognition half-site,” “recognition sequence half-site,” or simply “half-site” means a nucleic acid sequence in a double-stranded DNA molecule which is recognized by a monomer of a homodimeric or heterodimeric meganuclease, or by one subunit of a single-chain meganuclease.

As used herein, the term “hypervariable region” refers to a localized sequence within a meganuclease monomer or subunit that comprises amino acids with relatively high variability. A hypervariable region can comprise about 50-60 contiguous residues, about 53-57 contiguous residues, or preferably about 56 residues. In some embodiments, the residues of a hypervariable region may correspond to positions 24-79 or positions 215-270 of any one of SEQ ID NOs: 7-10. A hypervariable region can comprise one or more residues that contact DNA bases in a recognition sequence and can be modified to alter base preference of the monomer or subunit. A hypervariable region can also comprise one or more residues that bind to the DNA backbone when the meganuclease associates with a double-stranded DNA recognition sequence. Such residues can be modified to alter the binding affinity of the meganuclease for the DNA backbone and the target recognition sequence. In different embodiments of the invention, a hypervariable region may comprise between 1-20 residues that exhibit variability and can be modified to influence base preference and/or DNA-binding affinity. In particular embodiments, a hypervariable region comprises between about 15-20 residues that exhibit variability and can be modified to influence base preference and/or DNA-binding affinity.

In some embodiments, variable residues within a hypervariable region correspond to one or more of positions 24, 26, 28, 30, 32, 33, 38, 40, 42, 44, 46, 68, 70, 75, and 77 of any one of SEQ ID NOs: 7-10. In some embodiments, variable residues within a hypervariable region further correspond to position 37 of SEQ ID NO: 7. In some embodiments, variable residues within a hypervariable region further correspond to position 59 of SEQ ID NO: 10. In some embodiments, variable residues within a hypervariable region further correspond to one or more of positions 48, 50, 71, 72, or 73 of any one of SEQ ID NOs: 7-10.

In other embodiments, variable residues within a hypervariable region correspond to one or more of positions 215, 217, 219, 221, 223, 224, 229, 231, 233, 235, 237, 259, 261, 266, and 268 of any one of SEQ ID NOs: 7-10.

As used herein, the terms “T cell receptor alpha gene” or “TCR alpha gene” are interchangeable and refer to the locus in a T cell which encodes the T cell receptor alpha subunit. The T cell receptor alpha can refer to NCBI gene ID number 6955, before or after rearrangement. Following rearrangement, the T cell receptor alpha gene comprises an endogenous promoter, rearranged V and J segments, the endogenous splice donor site, an intron, the endogenous splice acceptor site, and the T cell receptor alpha constant region locus, which comprises the subunit coding exons.

As used herein, the term “T cell receptor alpha constant region” or “TCR alpha constant region” refers to the coding sequence of the T cell receptor alpha gene. The TCR alpha constant region includes the wild-type sequence, and functional variants thereof, identified by NCBI Gen ID NO. 28755.

The terms “recombinant DNA construct,” “recombinant construct,” “expression cassette,” “expression construct,” “chimeric construct,” “construct,” and “recombinant DNA fragment” are used interchangeably herein and are single or double-stranded polynucleotides. A recombinant construct comprises an artificial combination of nucleic acid fragments, including, without limitation, regulatory and coding sequences that are not found together in nature. For example, a recombinant DNA construct may comprise regulatory sequences and coding sequences that are derived from different sources, or regulatory sequences and coding sequences derived from the same source and arranged in a manner different than that found in nature. Such a construct may be used by itself or may be used in conjunction with a vector.

As used herein, a “human T cell” or “T cell” refers to a T cell isolated from a donor, particularly a human donor. T cells, and cells derived therefrom, include isolated T cells that have not been passaged in culture, T cells that have been passaged and maintained under cell culture conditions without immortalization, and T cells that have been immortalized and can be maintained under cell culture conditions indefinitely.

As used herein, a “human NK cell” or “NK cell” refers to a NK cell isolated from a donor, particularly a human donor. NK cells, and cells derived therefrom, include isolated NK cells that have not been passaged in culture, NK cells that have been passaged and maintained under cell culture conditions without immortalization, and NK cells that have been immortalized and can be maintained under cell culture conditions indefinitely.

As used herein, a “control” or “control cell” refers to a cell that provides a reference point for measuring changes in genotype or phenotype of a genetically-modified cell. A control cell may comprise, for example: (a) a wild-type cell, i.e., of the same genotype as the starting material for the genetic alteration which resulted in the genetically-modified cell; (b) a cell of the same genotype as the genetically-modified cell but which has been transformed with a null construct (i.e., with a construct which has no known effect on the trait of interest); or, (c) a cell genetically identical to the genetically-modified cell but which is not exposed to conditions or stimuli or further genetic modifications that would induce expression of altered genotype or phenotype.

As used herein, the terms “treatment” or “treating a subject” refers to the administration of a genetically-modified T cell or population of genetically-modified T cells of the invention to a subject having a disease. For example, the subject can have a disease such as cancer, and treatment can represent immunotherapy for the treatment of the disease. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In some aspects, a genetically-modified eukaryotic cell or population of genetically-modified eukaryotic cells described herein is administered during treatment in the form of a pharmaceutical composition of the invention.

The term “effective amount” or “therapeutically effective amount” refers to an amount sufficient to effect beneficial or desirable biological and/or clinical results. The therapeutically effective amount will vary depending on the formulation or composition used, the disease and its severity and the age, weight, physical condition and responsiveness of the subject to be treated. In specific embodiments, an effective amount of a genetically-modified T cell or population of genetically-modified T cells of the invention, or pharmaceutical compositions disclosed herein, reduces at least one symptom of a disease in a subject. In those embodiments wherein the disease is a cancer, an effective amount of the engineered meganuclease or pharmaceutical compositions disclosed herein reduces the level of proliferation or metastasis of cancer, causes a partial or full response or remission of cancer, or reduces at least one symptom of cancer in a subject.

As used herein, the term “cancer” should be understood to encompass any neoplastic disease (whether invasive or metastatic) which is characterized by abnormal and uncontrolled cell division causing malignant growth or tumor.

As used herein, the term “carcinoma” refers to a malignant growth made up of epithelial cells.

As used herein, the term “leukemia” refers to malignancies of the hematopoietic organs/systems and is generally characterized by an abnormal proliferation and development of leukocytes and their precursors in the blood and bone marrow.

As used herein, the term “sarcoma” refers to a tumor which is made up of a substance like the embryonic connective tissue and is generally composed of closely packed cells embedded in a fibrillary, heterogeneous, or homogeneous substance.

As used herein, the term “melanoma” refers to a tumor arising from the melanocytic system of the skin and other organs.

As used herein, the term “lymphoma” refers to a group of blood cell tumors that develop from lymphocytes.

As used herein, the term “blastoma” refers to a type of cancer that is caused by malignancies in precursor cells or blasts (immature or embryonic tissue).

As used herein, the recitation of a numerical range for a variable is intended to convey that the invention may be practiced with the variable equal to any of the values within that range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≥0 and ≤2 if the variable is inherently continuous.

### 2.1 Principle of the Invention

The present invention is based, in part, on the discovery of optimized, third-generation meganucleases that have improved properties compared to parental, first- and second-generation meganucleases, such as increased specificity, increased on-target activity, and reduced off-target cutting.

Like the previously described TRC 1-2x.87EE and TRC 1-2L.1592 meganucleases, these optimized, third-generation meganucleases recognize the TRC 1-2 recognition sequence (SEQ ID NO: 5) in exon 1 of the TCR alpha constant region gene. Cleavage at this recognition sequence can allow for NHEJ at the cleavage site and disrupted expression of the human T cell receptor alpha chain subunit, leading to reduced expression and/or function of the T cell receptor at the cell surface. Additionally, cleavage at this recognition sequence can further allow for homologous recombination of exogenous nucleic acid sequences directly into the TCR alpha constant region gene. Such exogenous nucleic acid sequences can comprise a sequence of interest, such as a sequence encoding a chimeric antigen receptor, an exogenous TCR receptor, or any other polypeptide of interest. Thus, the presently disclosed compositions and methods allow for both the knockout of the endogenous T cell receptor (e.g., an alpha/beta T cell receptor) and the expression of an exogenous nucleic acid sequence (e.g., a chimeric antigen receptor or exogenous TCR). Such cells can exhibit reduced or no induction of graft-versus-host-disease (GVHD) when administered to an allogeneic subject. However, the engineered meganucleases described herein exhibit improved characteristics including, for example, improved on-target activity (e.g., lower EC50 and EC90 values), reduced off-target activity, greater expansion of CAR T cells, and improved TRAC KO/CAR knock-in ratios of CAR T populations, as compared to previously disclosed TRC 1-2 meganucleases.

2.2 Optimized Meganucleases that Recognize and Cleave the TRC 1-2 Recognition Sequence within the T Cell Receptor Alpha Constant Region Gene

### Recognition Sequences

It is known in the art that it is possible to use a site-specific nuclease to make a DNA break in the genome of a living cell, and that such a DNA break can result in permanent modification of the genome via mutagenic NHEJ repair or via homologous recombination with a transgenic DNA sequence. NHEJ can produce mutagenesis at the cleavage site, resulting in inactivation of the allele. NHEJ-associated mutagenesis may inactivate an allele via generation of early stop codons, frameshift mutations producing aberrant non-functional proteins, or could trigger mechanisms such as nonsense-mediated mRNA decay. The use of nucleases to induce mutagenesis via NHEJ can be used to target a specific mutation or a sequence present in a wild-type allele. Further, the use of nucleases to induce a double-strand break in a target locus is known to stimulate homologous recombination, particularly of transgenic DNA sequences flanked by sequences that are homologous to the genomic target. In this manner, exogenous polynucleotides can be inserted into a target locus. Such exogenous polynucleotides can encode any sequence or polypeptide of interest.

Optimized engineered meganucleases described herein have been designed to bind and cleave a TRC 1-2 recognition sequence (SEQ ID NO: 5). Exemplary meganucleases that bind and cleave the TRC 1-2 recognition sequence are provided in SEQ ID NOs: 7-10.

### Exemplary Engineered Meganucleases

Engineered meganucleases of the invention comprise a first subunit, comprising a first hypervariable (HVR1) region, and a second subunit, comprising a second hypervariable (HVR2) region. Further, the first subunit binds to a first recognition half-site in the recognition sequence (e.g., the TRC1 half-site), and the second subunit binds to a second recognition half-site in the recognition sequence (e.g., the TRC2 half-site).

In particular embodiments, the meganucleases used to practice the invention are single-chain meganucleases. A single-chain meganuclease comprises an N-terminal subunit and a C-terminal subunit joined by a linker peptide. Each of the two subunits recognizes and binds to half of the recognition sequence (i.e., a recognition half-site) and the site of DNA cleavage is at the middle of the recognition sequence near the interface of the two subunits. DNA strand breaks are offset by four base pairs such that DNA cleavage by a meganuclease generates a pair of four base pair, 3′ single-strand overhangs. In embodiments where the recombinant meganuclease is a single-chain meganuclease, the first and second subunits can be oriented such that the first subunit, which comprises the HVR1 region and binds the first half-site, is positioned as the N-terminal subunit, and the second subunit, which comprises the HVR2 region and binds the second half-site, is positioned as the C-terminal subunit. In alternative embodiments, the first and second subunits can be oriented such that the first subunit, which comprises the HVR1 region and binds the first half-site, is positioned as the C-terminal subunit, and the second subunit, which comprises the HVR2 region and binds the second half-site, is positioned as the N-terminal subunit.

As discussed, the meganucleases of the invention have been engineered to bind and cleave the TRC 1-2 recognition sequence (SEQ ID NO: 5). The TRC 1-2 recognition sequence is positioned within exon 1 of the T cell receptor alpha constant region gene. Such engineered meganucleases are collectively referred to herein as “TRC 1-2 meganucleases.”

Exemplary TRC 1-2 meganucleases are provided in Tables 1 and 2 and are further described below.

TABLE 1
Exemplary engineered meganucleases which recognize and
cleave the TCR 1-2 recognition sequence (SEQ ID NO: 5).

| | | TRC1 | TRC1 | | | TRC2 | TRC2 | | |
|---|---|---|---|---|---|---|---|---|---|
| | AA | Subunit | Subunit | HVR1 | | Subunit | Subunit | HVR2 | |
| Meganuclease | SEQ ID | Residues | SEQ ID | Residues | *HVR1 % | Residues | SEQ ID | Residues | *HVR2 % |
| TRC 1-2L.2307 | 7 | 198-344 | 11 | 215-270 | 100 | 7-153 | 15 | 24-79 | 100 |
| TRC 1-2L.2213 | 8 | 198-344 | 12 | 215-270 | 100 | 7-153 | 16 | 24-79 | 98.21 |
| TRC 1-2L.2231 | 9 | 198-344 | 13 | 215-270 | 100 | 7-153 | 17 | 24-79 | 98.21 |
| TRC 1-2L.2338 | 10 | 198-344 | 14 | 215-270 | 100 | 7-153 | 18 | 24-79 | 96.43 |

*“HVR1 %” and “HVR2 %” represent the amino acid sequence identity between the HVR1 and HVR2 regions, respectively, of each meganuclease and the HVR1 and HVR2 regions, respectively, of the TRC 1-2L.2307 meganuclease.

TABLE 2
Exemplary engineered meganucleases which recognize and
cleave the TCR 1-2 recognition sequence (SEQ ID NO: 5).

| | | TRC1 | TRC1 | *TRC1 | TRC2 | TRC2 | *TRC2 |
|---|---|---|---|---|---|---|---|
| | AA | Subunit | Subunit | Subunit | Subunit | Subunit | Subunit |
| Meganuclease | SEQ ID | Residues | SEQ ID | % | Residues | SEQ ID | % |
| TRC 1-2L.2307 | 7 | 198-344 | 11 | 100 | 7-153 | 15 | 100 |
| TRC 1-2L.2213 | 8 | 198-344 | 12 | 100 | 7-153 | 16 | 99.32 |
| TRC 1-2L.2231 | 9 | 198-344 | 13 | 100 | 7-153 | 17 | 98.64 |
| TRC 1-2L.2338 | 10 | 198-344 | 14 | 99.32 | 7-153 | 18 | 97.96 |

*“TRC1 Subunit %” and “TRC 2 Subunit %” represent the amino acid sequence identity between the TRC1-binding and TRC2-binding subunit regions of each meganuclease and the TRC1-binding and TRC2-binding subunit regions, respectively, of the TRC 1-2L.2307 meganuclease.

### TRC 1-2L.2307 (SEQ ID NO: 7)

In some embodiments, the HVR1 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 215-270 of SEQ ID NO: 7. In some embodiments, the HVR1 region comprises one or more residues corresponding to residues 215, 217, 219, 221, 223, 224, 229, 231, 233, 235, 237, 259, 261, 266, and 268 of SEQ ID NO: 7. In some embodiments, the HVR1 region comprises residues corresponding to residues 215, 217, 219, 221, 223, 224, 229, 231, 233, 235, 237, 259, 261, 266, and 268 of SEQ ID NO: 7. In some embodiments, the HVR1 region comprises Y, R, K, or D at a residue corresponding to residue 257 of SEQ ID NO: 7. In some embodiments, the HVR1 region comprises residues 215-270 of SEQ ID NO: 7.

In some embodiments, the first subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 198-344 of SEQ ID NO: 7. In some embodiments, the first subunit comprises a residue corresponding to residue 271 of SEQ ID NO: 7. In some embodiments, the first subunit comprises G, S, or A at a residue corresponding to residue 210 of SEQ ID NO: 7. In some embodiments, the first subunit comprises E, Q, or K at a residue corresponding to residue 271 of SEQ ID NO: 7. In some embodiments, the first subunit comprises residues 198-344 of SEQ ID NO: 7.

In some embodiments, the HVR2 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 24-79 of SEQ ID NO: 7. In some embodiments, the HVR2 region comprises one or more residues corresponding to residues 24, 26, 28, 30, 32, 33, 38, 40, 42, 44, 46, 68, 70, 75, and 77 of SEQ ID NO: 7. In some embodiments, the HVR2 region comprises residues corresponding to residues 24, 26, 28, 30, 32, 33, 38, 40, 42, 44, 46, 68, 70, 75, and 77 of SEQ ID NO: 7. In some embodiments, the HVR2 region comprises a residue corresponding to residue 37 of SEQ ID NO: 7. In some embodiments, the HVR2 region comprises a residue corresponding to residue 48 of SEQ ID NO: 7. In some embodiments, the HVR2 region comprises a residue corresponding to residue 50 of SEQ ID NO: 7. In some embodiments, the HVR2 region comprises a residue corresponding to residue 71 of SEQ ID NO: 7. In some embodiments, the HVR2 region comprises a residue corresponding to residue 72 of SEQ ID NO: 7. In some embodiments, the HVR2 region comprises a residue corresponding to residue 73 of SEQ ID NO: 7. In some embodiments, the HVR2 region comprises Y, R, K, or D at a residue corresponding to residue 257 of SEQ ID NO: 7. In some embodiments, the HVR2 region comprises residues 24-79 of SEQ ID NO: 7.

In some embodiments, the second subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 7-153 of SEQ ID NO: 7. In some embodiments, the second subunit comprises a residue corresponding to residue 19 of SEQ ID NO: 7. In some embodiments, the second subunit comprises a residue corresponding to residue 139 of SEQ ID NO: 7. In some embodiments, the second subunit comprises G, S, or A at a residue corresponding to residue 19 of SEQ ID NO: 7. In some embodiments, the second subunit comprises E, Q, or K at a residue corresponding to residue 80 of SEQ ID NO: 7. In some embodiments, the second subunit comprises residues 7-153 of SEQ ID NO: 7.

In some embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to SEQ ID NO: 7. In some embodiments, the engineered meganuclease comprises an amino acid sequence of SEQ ID NO: 7.

In some embodiments, the engineered meganuclease is encoded by a nucleic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to a nucleic acid sequence set forth in SEQ ID NO: 19. In some embodiments, the engineered meganuclease is encoded by a nucleic acid sequence set forth in SEQ ID NO: 19.

### TR (1-21.2213 (SEQ ID) NO: 8)

In some embodiments, the HVR1 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 215-270 of SEQ ID NO: 8. In some embodiments, the HVR1 region comprises one or more residues corresponding to residues 215, 217, 219, 221, 223, 224, 229, 231, 233, 235, 237, 259, 261, 266, and 268 of SEQ ID NO: 8. In some embodiments, the HVR1 region comprises residues corresponding to residues 215, 217, 219, 221, 223, 224, 229, 231, 233, 235, 237, 259, 261, 266, and 268 of SEQ ID NO: 8. In some embodiments, the HVR1 region comprises Y, R, K, or D at a residue corresponding to residue 257 of SEQ ID NO: 8. In some embodiments, the HVR1 region comprises residues 215-270 of SEQ ID NO: 8.

In some embodiments, the first subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 198-344 of SEQ ID NO: 8. In some embodiments, the first subunit comprises a residue corresponding to residue 271 of SEQ ID NO: 8. In some embodiments, the first subunit comprises G, S, or A at a residue corresponding to residue 210 of SEQ ID NO: 8. In some embodiments, the first subunit comprises E, Q, or K at a residue corresponding to residue 271 of SEQ ID NO: 8. In some embodiments, the first subunit comprises residues 198-344 of SEQ ID NO: 8.

In some embodiments, the HVR2 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 24-79 of SEQ ID NO: 8. In some embodiments, the HVR2 region comprises one or more residues corresponding to residues 24, 26, 28, 30, 32, 33, 38, 40, 42, 44, 46, 68, 70, 75, and 77 of SEQ ID NO: 8. In some embodiments, the HVR2 region comprises residues corresponding to residues 24, 26, 28, 30, 32, 33, 38, 40, 42, 44, 46, 68, 70, 75, and 77 of SEQ ID NO: 8. In some embodiments, the HVR2 region comprises a residue corresponding to residue 48 of SEQ ID NO: 8. In some embodiments, the HVR2 region comprises a residue corresponding to residue 50 of SEQ ID NO: 8. In some embodiments, the HVR2 region comprises a residue corresponding to residue 71 of SEQ ID NO: 8. In some embodiments, the HVR2 region comprises a residue corresponding to residue 72 of SEQ ID NO: 8. In some embodiments, the HVR2 region comprises a residue corresponding to residue 73 of SEQ ID NO: 8. In some embodiments, the HVR2 region comprises Y, R, K, or D at a residue corresponding to residue 257 of SEQ ID NO: 8. In some embodiments, the HVR2 region comprises residues 24-79 of SEQ ID NO: 8.

In some embodiments, the second subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 7-153 of SEQ ID NO: 8. In some embodiments, the second subunit comprises a residue corresponding to residue 19 of SEQ ID NO: 8. In some embodiments, the second subunit comprises a residue corresponding to residue 139 of SEQ ID NO: 8. In some embodiments, the second subunit comprises G, S, or A at a residue corresponding to residue 19 of SEQ ID NO: 8. In some embodiments, the second subunit comprises E, Q, or K at a residue corresponding to residue 80 of SEQ ID NO: 8. In some embodiments, the second subunit comprises residues 7-153 of SEQ ID NO: 8.

In some embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to SEQ ID NO: 8. In some embodiments, the engineered meganuclease comprises an amino acid sequence of SEQ ID NO: 8.

In some embodiments, the engineered meganuclease is encoded by a nucleic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to a nucleic acid sequence set forth in SEQ ID NO: 20. In some embodiments, the engineered meganuclease is encoded by a nucleic acid sequence set forth in SEQ ID NO: 20.

### TRC 1-21.2231 (SEQ ID NO: 9)

In some embodiments, the HVR1 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 215-270 of SEQ ID NO: 9. In some embodiments, the HVR1 region comprises one or more residues corresponding to residues 215, 217, 219, 221, 223, 224, 229, 231, 233, 235, 237, 259, 261, 266, and 268 of SEQ ID NO: 9. In some embodiments, the HVR1 region comprises residues corresponding to residues 215, 217, 219, 221, 223, 224, 229, 231, 233, 235, 237, 259, 261, 266, and 268 of SEQ ID NO: 9. In some embodiments, the HVR1 region comprises Y, R, K, or D at a residue corresponding to residue 257 of SEQ ID NO: 9. In some embodiments, the HVR1 region comprises residues 215-270 of SEQ ID NO: 9.

In some embodiments, the first subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 198-344 of SEQ ID NO: 9. In some embodiments, the first subunit comprises a residue corresponding to residue 271 of SEQ ID NO: 9. In some embodiments, the first subunit comprises G, S, or A at a residue corresponding to residue 210 of SEQ ID NO: 9. In some embodiments, the first subunit comprises E, Q, or K at a residue corresponding to residue 271 of SEQ ID NO: 9. In some embodiments, the first subunit comprises residues 198-344 of SEQ ID NO: 9.

In some embodiments, the HVR2 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 24-79 of SEQ ID NO: 9. In some embodiments, the HVR2 region comprises one or more residues corresponding to residues 24, 26, 28, 30, 32, 33, 38, 40, 42, 44, 46, 68, 70, 75, and 77 of SEQ ID NO: 9. In some embodiments, the HVR2 region comprises residues corresponding to residues 24, 26, 28, 30, 32, 33, 38, 40, 42, 44, 46, 68, 70, 75, and 77 of SEQ ID NO: 9. In some embodiments, the HVR2 region comprises a residue corresponding to residue 48 of SEQ ID NO: 9. In some embodiments, the HVR2 region comprises a residue corresponding to residue 50 of SEQ ID NO: 9. In some embodiments, the HVR2 region comprises a residue corresponding to residue 71 of SEQ ID NO: 9. In some embodiments, the HVR2 region comprises a residue corresponding to residue 72 of SEQ ID NO: 9. In some embodiments, the HVR2 region comprises a residue corresponding to residue 73 of SEQ ID NO: 9. In some embodiments, the HVR2 region comprises Y, R, K, or D at a residue corresponding to residue 257 of SEQ ID NO: 9. In some embodiments, the HVR2 region comprises residues 24-79 of SEQ ID NO: 9.

In some embodiments, the second subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 7-153 of SEQ ID NO: 9. In some embodiments, the second subunit comprises a residue corresponding to residue 19 of SEQ ID NO: 9. In some embodiments, the second subunit comprises a residue corresponding to residue 80 of SEQ ID NO: 9. In some embodiments, the second subunit comprises a residue corresponding to residue 139 of SEQ ID NO: 9. In some embodiments, the second subunit comprises G, S, or A at a residue corresponding to residue 19 of SEQ ID NO: 9. In some embodiments, the second subunit comprises E, Q, or K at a residue corresponding to residue 80 of SEQ ID NO: 9. In some embodiments, the second subunit comprises residues 7-153 of SEQ ID NO: 9.

In some embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to SEQ ID NO: 9. In some embodiments, the engineered meganuclease comprises an amino acid sequence of SEQ ID NO: 9.

In some embodiments, the engineered meganuclease is encoded by a nucleic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to a nucleic acid sequence set forth in SEQ ID NO: 21. In some embodiments, the engineered meganuclease is encoded by a nucleic acid sequence set forth in SEQ ID NO: 21.

### TRC 1-21.2338 (SEQ ID) NO: 10)

In some embodiments, the HVR1 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 215-270 of SEQ ID NO: 10. In some embodiments, the HVR1 region comprises one or more residues corresponding to residues 215, 217, 219, 221, 223, 224, 229, 231, 233, 235, 237, 259, 261, 266, and 268 of SEQ ID NO: 10. In some embodiments, the HVR1 region comprises residues corresponding to residues 215, 217, 219, 221, 223, 224, 229, 231, 233, 235, 237, 259, 261, 266, and 268 of SEQ ID NO: 10. In some embodiments, the HVR1 region comprises Y, R, K, or D at a residue corresponding to residue 257 of SEQ ID NO: 10. In some embodiments, the HVR1 region comprises residues 215-270 of SEQ ID NO: 10.

In some embodiments, the first subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 198-344 of SEQ ID NO: 10. In some embodiments, the first subunit comprises G, S, or A at a residue corresponding to residue 210 of SEQ ID NO: 10. In some embodiments, the first subunit comprises E, Q, or K at a residue corresponding to residue 271 of SEQ ID NO: 10. In some embodiments, the first subunit comprises residues 198-344 of SEQ ID NO: 10.

In some embodiments, the HVR2 region comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to an amino acid sequence corresponding to residues 24-79 of SEQ ID NO: 10. In some embodiments, the HVR2 region comprises one or more residues corresponding to residues 24, 26, 28, 30, 32, 33, 38, 40, 42, 44, 46, 68, 70, 75, and 77 of SEQ ID NO: 10. In some embodiments, the HVR2 region comprises residues corresponding to residues 24, 26, 28, 30, 32, 33, 38, 40, 42, 44, 46, 68, 70, 75, and 77 of SEQ ID NO: 10. In some embodiments, the HVR2 region comprises a residue corresponding to residue 48 of SEQ ID NO: 10. In some embodiments, the HVR2 region comprises a residue corresponding to residue 50 of SEQ ID NO: 10. In some embodiments, the HVR2 region comprises a residue corresponding to residue 59 of SEQ ID NO: 10. In some embodiments, the HVR2 region comprises a residue corresponding to residue 71 of SEQ ID NO: 10. In some embodiments, the HVR2 region comprises a residue corresponding to residue 72 of SEQ ID NO: 10. In some embodiments, the HVR2 region comprises a residue corresponding to residue 73 of SEQ ID NO: 10. In some embodiments, the HVR2 region comprises Y, R, K, or D at a residue corresponding to residue 257 of SEQ ID NO: 10. In some embodiments, the HVR2 region comprises residues 24-79 of SEQ ID NO: 10.

In some embodiments, the second subunit comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to residues 7-153 of SEQ ID NO: 10. In some embodiments, the second subunit comprises a residue corresponding to residue 19 of SEQ ID NO: 10. In some embodiments, the second subunit comprises a residue corresponding to residue 80 of SEQ ID NO: 10. In some embodiments, the second subunit comprises a residue corresponding to residue 139 of SEQ ID NO: 10. In some embodiments, the second subunit comprises G, S, or A at a residue corresponding to residue 19 of SEQ ID NO: 10. In some embodiments, the second subunit comprises E, Q, or K at a residue corresponding to residue 80 of SEQ ID NO: 10. In some embodiments, the second subunit comprises residues 7-153 of SEQ ID NO: 10.

In some embodiments, the engineered meganuclease is a single-chain meganuclease comprising a linker, wherein the linker covalently joins the first subunit and the second subunit.

In some embodiments, the engineered meganuclease comprises an amino acid sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to SEQ ID NO: 10. In some embodiments, the engineered meganuclease comprises an amino acid sequence of SEQ ID NO: 10.

In some embodiments, the engineered meganuclease is encoded by a nucleic sequence having at least 80%, at least 85%, at least 86%, at least 87%, at least 88%, at least 89%, at least 90%, at least 91%, at least 92%, at least 93%, at least 94%, at least 95%, at least 96%, at least 97%, at least 98% at least 99%, or more, sequence identity to a nucleic acid sequence set forth in SEQ ID NO: 22. In some embodiments, the engineered meganuclease is encoded by a nucleic acid sequence set forth SEQ ID NO: 22.

### 2.3 Methods for Delivering Optimized Meganucleases

The invention provides methods for producing genetically-modified eukaryotic cells (e.g., T cell, NK cells, iPSCs) and populations thereof using engineered meganucleases that recognize and cleave recognition sequences found within the human TCR alpha constant region gene (SEQ ID NO: 3). Immune cells, such as T cells or NK cells, can be obtained from any number of sources, including peripheral blood mononuclear cells, bone marrow, lymph node tissue, cord blood, thymus tissue, tissue from a site of infection, ascites, pleural effusion, spleen tissue, and tumors. In certain embodiments of the present disclosure, any number of cell lines available in the art may be used. In some embodiments of the present disclosure, eukaryotic cells such as T cells or NK cells are obtained from a unit of blood collected from a subject using any number of techniques known to the skilled artisan. In one embodiment, cells from the circulating blood of an individual are obtained by apheresis.

The engineered meganucleases described herein are capable of generating a modified T cell receptor alpha constant region gene, specifically in exon 1 where the TRC 1-2 recognition sequence is located, either through introduction of an insertion or deletion via non-homologous end joining, or through introduction of a donor template encoding an exogenous sequence of interest (e.g., encoding a transgene).

As used herein, the term “exogenous” or “heterologous” in reference to a nucleotide sequence is intended to mean a sequence that is purely synthetic, that originates from a foreign species, or, if from the same species, is substantially modified from its native form in composition and/or genomic locus by deliberate human intervention.

In various embodiments, the exogenous sequence of interest can comprise a coding sequence for a protein of interest. It is envisioned that the coding sequence can be for any protein of interest.

In certain embodiments, the exogenous sequence of interest comprises a nucleic acid sequence encoding a chimeric antigen receptor (CAR). Generally, a CAR of the present disclosure will comprise at least an extracellular domain and an intracellular domain. In some embodiments, the extracellular domain comprises a target-specific binding element otherwise referred to as a ligand-binding domain or moiety. In some embodiments, the intracellular domain, or cytoplasmic domain, comprises at least one co-stimulatory domain and one or more signaling domains such as, for example, CD3ζ.

In some embodiments, a CAR comprises an extracellular, target-specific binding element otherwise referred to as a ligand-binding domain or moiety. The choice of ligand-binding domain depends upon the type and number of ligands that define the surface of a target cell. For example, the ligand-binding domain may be chosen to recognize a ligand that acts as a cell surface marker on target cells associated with a particular disease state. Thus, examples of cell surface markers that may act as ligands for the ligand-binding domain in a CAR can include those associated with viruses, bacterial and parasitic infections, autoimmune disease, and cancer cells. In some embodiments, a CAR is engineered to target a tumor-specific antigen of interest by way of engineering a desired ligand-binding moiety that specifically binds to an antigen on a tumor cell. In the context of the present disclosure, “tumor antigen” or “tumor-specific antigen” refer to antigens that are common to specific hyperproliferative disorders such as cancer.

In some embodiments, the extracellular ligand-binding domain of the CAR is specific for any antigen or epitope of interest, particularly any tumor antigen or epitope of interest. As non-limiting examples, in some embodiments the antigen of the target is a tumor-associated surface antigen, such as ErbB2 (HER2/neu), carcinoembryonic antigen (CEA), epithelial cell adhesion molecule (EpCAM), epidermal growth factor receptor (EGFR), EGFR variant III (EGFRvIII), CD19, CD20, CD22, CD30, CD40, CD79B, IL1RAP, glypican 3 (GPC3), CLL-1, disialoganglioside GD2, ductal-epithelial mucine, gp36, TAG-72, glycosphingolipids, glioma-associated antigen, B-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostase specific antigen (PSA), PAP, NY-ESO-1, LAGA-la, p53, prostein, PSMA, surviving and telomerase, prostate-carcinoma tumor antigen-1 (PCTA-1), MAGE, ELF2M, neutrophil elastase, ephrin B2, insulin growth factor (IGF1)-1, IGF-II, IGFI receptor, mesothelin, a major histocompatibility complex (MHC) molecule presenting a tumor-specific peptide epitope, 5T4, ROR1, Nkp30, NKG2D, tumor stromal antigens, the extra domain A (EDA) and extra domain B (EDB) of fibronectin and the Al domain of tenascin-C (TnC Al) and fibroblast associated protein (fap); a lineage-specific or tissue specific antigen such as CD3, CD4, CD8, CD24, CD25, CD33, CD34, CD38, CD123, CD133, CD138, CTLA-4, B7-1 (CD80), B7-2 (CD86), endoglin, a major histocompatibility complex (MHC) molecule, BCMA (CD269, TNFRSF 17), CS1, or a virus-specific surface antigen such as an HIV-specific antigen (such as HIV gpl20); an EBV-specific antigen, a CMV-specific antigen, a HPV-specific antigen such as the E6 or E7 oncoproteins, a Lasse Virus-specific antigen, an Influenza Virus-specific antigen, as well as any derivate or variant of these surface markers.

In some examples, the extracellular ligand-binding domain or moiety is an antibody, or antibody fragment. An antibody fragment can, for example, be at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (Fn3) (see U.S. Pat. No. 6,703,199, which describes fibronectin polypeptide minibodies).

In some embodiments, the extracellular ligand-binding domain or moiety is in the form of a single-chain variable fragment (scFv) derived from a monoclonal antibody, which provides specificity for a particular epitope or antigen (e.g., an epitope or antigen preferentially present on the surface of a cell, such as a cancer cell or other disease-causing cell or particle). In some such embodiments, the scFv can comprise a heavy chain variable (VH) domain and a light chain variable (VL) domain from a monoclonal antibody having specificity for an antigen. In some embodiments, the scFv is attached via a linker sequence. In some embodiments, the scFv is murine, humanized, or fully human.

In some embodiments, the extracellular domain of a chimeric antigen receptor comprises an autoantigen (see, Payne et al. (2016) Science, Vol. 353 (6295): 179-184), which can be recognized by autoantigen-specific B cell receptors on B lymphocytes, thus directing T cells to specifically target and kill autoreactive B lymphocytes in antibody-mediated autoimmune diseases. Such CARs can be referred to as chimeric autoantibody receptors (CAARs).

In some embodiments, the extracellular domain of a chimeric antigen receptor can comprise a naturally-occurring ligand for an antigen of interest, or a fragment of a naturally-occurring ligand which retains the ability to bind the antigen of interest.

In some embodiments, a CAR comprises a transmembrane domain which links the extracellular ligand-binding domain or autoantigen with the intracellular signaling and co-stimulatory domains via a hinge or spacer sequence. The transmembrane domain can be derived from any membrane-bound or transmembrane protein. For example, the transmembrane polypeptide can be a subunit of the T-cell receptor (i.e., an α, β, γ or ζ, polypeptide constituting CD3 complex), IL2 receptor p55 (a chain), p75 (β chain) or γ chain, subunit chain of Fc receptors (e.g., Fcy receptor III) or CD proteins such as the CD8 alpha chain. Alternatively, the transmembrane domain can be synthetic and can comprise predominantly hydrophobic residues such as leucine and valine. In particular examples, the transmembrane domain is a CD8α transmembrane polypeptide.

The hinge region refers to any oligo- or polypeptide that functions to link the transmembrane domain to the extracellular ligand-binding domain. For example, a hinge region may comprise up to 300 amino acids, preferably 10 to 100 amino acids and most preferably 25 to 50 amino acids. Hinge regions may be derived from all or part of naturally occurring molecules, such as from all or part of the extracellular region of CD8, CD4 or CD28, or from all or part of an antibody constant region. Alternatively, the hinge region may be a synthetic sequence that corresponds to a naturally occurring hinge sequence, or may be an entirely synthetic hinge sequence. In particular examples, a hinge domain can comprise a part of a human CD8 alpha chain, FcyRIIIa receptor, or IgGI. In certain examples, the hinge region can be a CD8 alpha domain.

Intracellular signaling domains of a CAR are responsible for activation of at least one of the normal effector functions of the cell in which the CAR has been placed and/or activation of proliferative and cell survival pathways. The term “effector function” refers to a specialized function of a cell. Effector function of a T cell, for example, may be cytolytic activity or helper activity including the secretion of cytokines. An intracellular signaling domain, such as CD3ζ, can provide an activation signal to the cell in response to binding of the extracellular domain. As discussed, the activation signal can induce an effector function of the cell such as, for example, cytolytic activity or cytokine secretion.

The intracellular stimulatory domain can also include one or more intracellular co-stimulatory domains that transmit a proliferative and/or cell-survival signal after ligand binding. In some cases, the co-stimulatory domain can comprise one or more TRAF-binding domains. Such intracellular co-stimulatory domains can be any of those known in the art and can include, without limitation, those co-stimulatory domains disclosed in WO 2018/067697 including, for example, Novel 6 (“N6”). Further examples of co-stimulatory domains can include 4-1BB (CD137), CD27, CD28, CD8, OX40, CD30, CD40, PD-1, ICOS, lymphocyte function-associated antigen-1 (LFA-1), CD2, CD7, LIGHT, NKG2C, B7-H3, a ligand that specifically binds with CD83, or any combination thereof. In a particular embodiment, the co-stimulatory domain is an N6 domain. In another particular embodiment, the co-stimulatory domain is a 4-1BB co-stimulatory domain.

In other embodiments, the genetically-modified immune cell comprises a nucleic acid sequence encoding an exogenous T cell receptor (TCR). Such exogenous T cell receptors can comprise alpha and beta chains or, alternatively, may comprise gamma and delta chains. Exogenous TCRs useful in the invention may have specificity to any antigen or epitope of interest such as, without limitation, any antigen or epitope disclosed herein.

The CAR or exogenous TCR can be specific for any type of cancer cell. Such cancers can include, without limitation, carcinoma, lymphoma, sarcoma, blastomas, leukemia, cancers of B cell origin, breast cancer, gastric cancer, neuroblastoma, osteosarcoma, lung cancer, melanoma, prostate cancer, colon cancer, renal cell carcinoma, ovarian cancer, rhabdomyosarcoma, leukemia, and Hodgkin lymphoma. In specific embodiments, cancers and disorders include but are not limited to pre-B ALL (pediatric indication), adult ALL, mantle cell lymphoma, diffuse large B cell lymphoma, salvage post allogenic bone marrow transplantation, and the like. These cancers can be treated using a combination of CARs or exogenous TCRs that target, for example, CD19, CD20, CD22, and/or ROR1. In some non-limiting examples, a genetically-modified immune cell or population thereof of the present disclosure targets carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, multiple myeloma, and B-cell non-Hodgkin lymphoma. In some examples, cancers can include, without limitation, cancers of B cell origin or multiple myeloma. In some examples, the cancer of B cell origin is acute lymphoblastic leukemia (ALL), chronic lymphocytic leukemia (CLL), small lymphocytic lymphoma (SLL), or non-Hodgkin lymphoma (NHL). In some examples, the cancer of B cell origin is mantle cell lymphoma (MCL) or diffuse large B cell lymphoma (DLBCL).

In other embodiments, the sequence of interest can encode a wild-type or modified version of an endogenous gene of interest.

The sequence of interest can comprise an element or peptide known in the art to allow for the translation of two more genes from the same promoter, including but not limited to IRES elements and 2A elements, such as, a T2A element, a P2A element, an E2A element, and an F2A element. In specific embodiments, such elements in the exogenous sequence of interest can be located 5′ upstream, or 3′ downstream of a nucleic acid sequence encoding a protein of interest.

The exogenous sequence of interest described herein can further comprise additional control sequences. For example, the sequences of interest can include homologous recombination enhancer sequences, Kozak sequences, polyadenylation sequences, transcriptional termination sequences, selectable marker sequences (e.g., antibiotic resistance genes), origins of replication, and the like. Sequences of interest described herein can also include at least one nuclear localization signal. Examples of nuclear localization signals are known in the art (see, e.g., Lange et al., J. Biol. Chem., 2007, 282:5101-5105).

Engineered meganucleases described herein can be delivered into a cell in the form of protein or, preferably, as a nucleic acid encoding the engineered meganuclease. Such nucleic acid can be DNA (e.g., circular or linearized plasmid DNA or PCR products) or RNA (e.g., mRNA). For embodiments in which the engineered meganuclease coding sequence is delivered in DNA form, it should be operably linked to a promoter to facilitate transcription of the meganuclease gene. Mammalian promoters suitable for the invention include constitutive promoters such as the cytomegalovirus early (CMV) promoter (Thomsen et al. (1984), Proc Natl Acad Sci USA. 81 (3): 659-63) or the SV40 early promoter (Benoist and Chambon (1981), Nature. 290 (5804): 304-10) as well as inducible promoters such as the tetracycline-inducible promoter (Dingermann et al. (1992), Mol Cell Biol. 12 (9): 4038-45). An engineered meganuclease of the invention can also be operably linked to a synthetic promoter. Synthetic promoters can include, without limitation, the JeT promoter (WO 2002/012514).

In some embodiments, mRNA encoding the engineered meganuclease is delivered to the cell because this reduces the likelihood that the gene encoding the engineered meganuclease will integrate into the genome of the cell. Such mRNA encoding an engineered meganuclease can be produced using methods known in the art such as in vitro transcription. In some embodiments, the mRNA is 5′ capped using 7-methyl-guanosine, anti-reverse cap analogs (ARCA) (U.S. Pat. No. 7,074,596), CleanCap® analogs such as Cap 1 analogs (Trilink, San Diego, CA), or enzymatically capped using vaccinia capping enzyme or similar. In some embodiments, the mRNA may be polyadenylated. The mRNA may contain various 5′ and 3′ untranslated sequence elements to enhance expression the encoded engineered meganuclease and/or stability of the mRNA itself. Such elements can include, for example, posttranslational regulatory elements such as a woodchuck hepatitis virus posttranslational regulatory element. The mRNA may contain nucleoside analogs or naturally-occurring nucleosides, such as pseudouridine, 5-methylcytidine, N6-methyladenosine, 5-methyluridine, or 2-thiouridine. Additional nucleoside analogs include, for example, those described in U.S. Pat. No. 8,278,036.

In particular embodiments, an mRNA encoding an engineered meganuclease of the invention can be a polycistronic mRNA encoding two or more meganucleases that are simultaneously expressed in the cell. A polycistronic mRNA can encode two or more meganucleases that target different recognition sequences in the same target gene. Alternatively, a polycistronic mRNA can encode at least one meganuclease described herein and at least one additional nuclease targeting a separate recognition sequence positioned in the same gene, or targeting a second recognition sequence positioned in a second gene such that cleavage sites are produced in both genes. A polycistronic mRNA can comprise any element known in the art to allow for the translation of two or more genes (i.e., cistrons) from the same mRNA molecule including, but not limited to, an IRES element, a T2A element, a P2A element, an E2A element, and an F2A element.

In another particular embodiment, a nucleic acid encoding an engineered meganuclease of the invention can be introduced into the cell using a single-stranded DNA template. The single-stranded DNA can further comprise a 5′ and/or a 3′ AAV inverted terminal repeat (ITR) upstream and/or downstream of the sequence encoding the engineered meganuclease. In other embodiments, the single-stranded DNA can further comprise a 5′ and/or a 3′ homology arm upstream and/or downstream of the sequence encoding the engineered meganuclease.

In another particular embodiment, genes encoding a meganuclease of the invention can be introduced into a cell using a linearized DNA template. In some examples, a plasmid DNA encoding a meganuclease can be digested by one or more restriction enzymes such that the circular plasmid DNA is linearized prior to being introduced into a cell.

Purified meganuclease proteins can be delivered into cells to cleave genomic DNA, which allows for homologous recombination or non-homologous end-joining at the cleavage site with a sequence of interest, by a variety of different mechanisms known in the art, including those further detailed herein below.

In some embodiments, meganuclease proteins, or DNA/mRNA encoding the meganuclease, are coupled to a cell penetrating peptide or targeting ligand to facilitate cellular uptake. Examples of cell penetrating peptides known in the art include poly-arginine (Jearawiriyapaisarn, et al. (2008) Mol Ther. 16:1624-9), TAT peptide from the HIV virus (Hudecz et al. (2005), Med. Res. Rev. 25:679-736), MPG (Simeoni, et al. (2003) Nucleic Acids Res. 31:2717-2724), Pep-1 (Deshayes et al. (2004) Biochemistry 43:7698-7706), and HSV-1 VP-22 (Deshayes et al. (2005) Cell Mol Life Sci. 62:1839-49). In an alternative embodiment, meganuclease proteins, or DNA/mRNA encoding meganucleases, are coupled covalently or non-covalently to an antibody that recognizes a specific cell-surface receptor expressed on target cells such that the meganuclease protein/DNA/mRNA binds to and is internalized by the target cells. Alternatively, meganuclease protein/DNA/mRNA can be coupled covalently or non-covalently to the natural ligand (or a portion of the natural ligand) for such a cell-surface receptor. (McCall, et al. (2014) Tissue Barriers. 2 (4): e944449; Dinda, et al. (2013) Curr Pharm Biotechnol. 14:1264-74; Kang, et al. (2014) Curr Pharm Biotechnol. 15 (3): 220-30; Qian et al. (2014) Expert Opin Drug Metab Toxicol. 10 (11): 1491-508).

In some embodiments, meganuclease proteins, or DNA/mRNA encoding meganucleases, are coupled covalently or non-covalently to a nanoparticle or encapsulated within such a nanoparticle using methods known in the art (Sharma, et al. (2014) Biomed Res Int. 2014). A nanoparticle is a nanoscale delivery system whose length scale is <1 μm, preferably <100 nm. Such nanoparticles may be designed using a core composed of metal, lipid, polymer, or biological macromolecule, and multiple copies of the recombinant meganuclease proteins, mRNA, or DNA can be attached to or encapsulated with the nanoparticle core. This increases the copy number of the protein/mRNA/DNA that is delivered to each cell and, so, increases the intracellular expression of each engineered meganuclease to maximize the likelihood that the target recognition sequences will be cut. The surface of such nanoparticles may be further modified with polymers or lipids (e.g., chitosan, cationic polymers, or cationic lipids) to form a core-shell nanoparticle whose surface confers additional functionalities to enhance cellular delivery and uptake of the payload (Jian et al. (2012) Biomaterials. 33 (30): 7621-30). Nanoparticles may additionally be advantageously coupled to targeting molecules to direct the nanoparticle to the appropriate cell type and/or increase the likelihood of cellular uptake. Examples of such targeting molecules include antibodies specific for cell surface receptors and the natural ligands (or portions of the natural ligands) for cell surface receptors.

In some embodiments, the meganuclease proteins or DNA/mRNA encoding the meganucleases are encapsulated within liposomes or complexed using cationic lipids (see, e.g., Lipofectamine™, Life Technologies Corp., Carlsbad, CA; Zuris et al. (2015) Nat Biotechnol. 33:73-80; Mishra et al. (2011) J Drug Deliv. 2011:863734). The liposome and lipoplex formulations can protect the payload from degradation, and facilitate cellular uptake and delivery efficiency through fusion with and/or disruption of the cellular membranes of the target cells.

In some embodiments, meganuclease proteins, or DNA/mRNA encoding meganucleases, are encapsulated within polymeric scaffolds (e.g., PLGA) or complexed using cationic polymers (e.g., PEI, PLL) (Tamboli et al. (2011) Ther Deliv. 2 (4): 523-536). Polymeric carriers can be designed to provide tunable drug release rates through control of polymer erosion and drug diffusion, and high drug encapsulation efficiencies can offer protection of the therapeutic payload until intracellular delivery to the desired target cell population.

In some embodiments, meganuclease proteins, or DNA/mRNA encoding recombinant meganucleases, are combined with amphiphilic molecules that self-assemble into micelles (Tong et al. (2007) J Gene Med. 9 (11): 956-66). Polymeric micelles may include a micellar shell formed with a hydrophilic polymer (e.g., polyethyleneglycol) that can prevent aggregation, mask charge interactions, and reduce nonspecific interactions.

In some embodiments, meganuclease proteins, or DNA/mRNA encoding meganucleases, are formulated into an emulsion or a nanoemulsion (i.e., having an average particle diameter of <1 nm) for administration and/or delivery to the target cell. The term “emulsion” refers to, without limitation, any oil-in-water, water-in-oil, water-in-oil-in-water, or oil-in-water-in-oil dispersions or droplets, including lipid structures that can form as a result of hydrophobic forces that drive apolar residues (e.g., long hydrocarbon chains) away from water and polar head groups toward water, when a water immiscible phase is mixed with an aqueous phase. These other lipid structures include, but are not limited to, unilamellar, paucilamellar, and multilamellar lipid vesicles, micelles, and lamellar phases. Emulsions are composed of an aqueous phase and a lipophilic phase (typically containing an oil and an organic solvent). Emulsions also frequently contain one or more surfactants. Nanoemulsion formulations are well known, e.g., as described in US Patent Application Nos. 2002/0045667 and 2004/0043041, and U.S. Pat. Nos. 6,015,832, 6,506,803, 6,635,676, and 6,559,189, each of which is incorporated herein by reference in its entirety.

In some embodiments, meganuclease proteins, or DNA/mRNA encoding meganucleases, are covalently attached to, or non-covalently associated with, multifunctional polymer conjugates, DNA dendrimers, and polymeric dendrimers (Mastorakos et al. (2015) Nanoscale. 7 (9): 3845-56; Cheng et al. (2008) J Pharm Sci. 97 (1): 123-43). The dendrimer generation can control the payload capacity and size, and can provide a high drug payload capacity. Moreover, display of multiple surface groups can be leveraged to improve stability, reduce nonspecific interactions, and enhance cell-specific targeting and drug release.

In some embodiments, genes encoding a meganuclease are delivered using a recombinant virus. Such recombinant viruses are known in the art and include retroviruses, lentiviruses, adenoviruses, and adeno-associated viruses (AAVs) (reviewed in Vannucci, et al. (2013 New Microbiol. 36:1-22). Recombinant AAVs useful in the invention can have any serotype that allows for transduction of the virus into the cell and insertion of the nuclease gene into the cell genome. In particular embodiments, recombinant AAVs have a serotype of AAV2 or AAV6. AAVs can also be self-complementary such that they do not require second-strand DNA synthesis in the host cell (McCarty, et al. (2001) Gene Ther. 8:1248-54).

If the meganuclease genes are delivered in DNA form (e.g., plasmid) and/or via a recombinant virus (e.g., AAV) they must be operably linked to a promoter. In some embodiments, this can be a viral promoter such as endogenous promoters from the viral vector (e.g., the LTR of a lentiviral vector) or the well-known cytomegalovirus- or SV40 virus-early promoters. In a preferred embodiment, meganuclease genes are operably linked to a promoter that drives gene expression preferentially in the target cell (e.g., a T cell).

The invention further provides for the introduction of an exogenous sequence of interest into the T cell receptor alpha constant region gene at the TRC 1-2 recognition sequence. In some embodiments, the exogenous sequence of interest comprises a 5′ homology arm and a 3′ homology arm flanking the elements of the insert. Such homology arms have sequence homology to corresponding sequences 5′ upstream and 3′ downstream of the nuclease recognition sequence where a cleavage site is produced. In general, homology arms can have a length of at least 50 base pairs, preferably at least 100 base pairs, and up to 2000 base pairs or more, and can have at least 90%, preferably at least 95%, or more, sequence homology to their corresponding sequences in the genome.

The exogenous sequence of interest of the invention may be introduced into the cell by any of the means previously discussed. In a particular embodiment, the exogenous sequence of interest is introduced by way of a recombinant viral, such as a lentivirus, retrovirus, adenovirus, or preferably a recombinant AAV. Recombinant AAVs useful for introducing an exogenous nucleic acid can have any serotype that allows for transduction of the virus into the cell and insertion of the exogenous nucleic acid sequence into the cell genome. In particular embodiments, the recombinant AAV has a serotype of AAV2 or AAV6. The recombinant AAV can also be self-complementary such that it do not require second-strand DNA synthesis in the host cell.

In another particular embodiment, the exogenous sequence of interest can be introduced into the cell using a single-stranded DNA template. The single-stranded DNA can comprise the exogenous sequence of interest and, in preferred embodiments, can comprise 5′ and 3′ homology arms to promote insertion of the nucleic acid sequence into the meganuclease cleavage site by homologous recombination. The single-stranded DNA can further comprise a 5′ AAV inverted terminal repeat (ITR) sequence 5′ upstream of the 5′ homology arm, and a 3′ AAV ITR sequence 3′ downstream of the 3′ homology arm.

In another particular embodiment, genes encoding an engineered meganuclease of the invention and/or an exogenous sequence of interest of the invention can be introduced into the cell by transfection with a linearized DNA template. In some examples, a plasmid DNA can be digested by one or more restriction enzymes such that the circular plasmid DNA is linearized prior to transfection into the cell.

T cells modified by the present invention may require activation prior to introduction of a meganuclease and/or an exogenous sequence of interest. For example, T cells can be contacted with anti-CD3 and anti-CD28 antibodies that are soluble or conjugated to a support (i.e., beads) for a period of time sufficient to activate the cells.

Genetically-modified eukaryotic cells of the invention can be further modified to express one or more inducible suicide genes, the induction of which provokes cell death and allows for selective destruction of the cells in vitro or in vivo. In some examples, a suicide gene can encode a cytotoxic polypeptide, a polypeptide that has the ability to convert a non-toxic pro-drug into a cytotoxic drug, and/or a polypeptide that activates a cytotoxic gene pathway within the cell. That is, a suicide gene is a nucleic acid that encodes a product that causes cell death by itself or in the presence of other compounds. A representative example of such a suicide gene is one that encodes thymidine kinase of herpes simplex virus. Additional examples are genes that encode thymidine kinase of varicella zoster virus and the bacterial gene cytosine deaminase that can convert 5-fluorocytosine to the highly toxic compound 5-fluorouracil. Suicide genes also include as non-limiting examples genes that encode caspase-9, caspase-8, or cytosine deaminase. In some examples, caspase-9 can be activated using a specific chemical inducer of dimerization (CID). A suicide gene can also encode a polypeptide that is expressed at the surface of the cell that makes the cells sensitive to therapeutic and/or cytotoxic monoclonal antibodies. In further examples, a suicide gene can encode recombinant antigenic polypeptide comprising an antigenic motif recognized by the anti-CD20 mAb Rituximab and an epitope that allows for selection of cells expressing the suicide gene. See, for example, the RQR8 polypeptide described in WO2013153391, which comprises two Rituximab-binding epitopes and a QBEnd10-binding epitope. For such a gene, Rituximab can be administered to a subject to induce cell depletion when needed. In further examples, a suicide gene may include a QBEnd10-binding epitope expressed in combination with a truncated EGFR polypeptide.

Eukaryotic cells modified by the methods and compositions described herein can exhibit no cell surface expression of expression of an endogenous alpha/beta T cell receptor and, optionally, can further express a protein of interest (e.g., a CAR). Thus, the invention further provides a population of eukaryotic cells that express the protein of interest and do not express the endogenous alpha/beta T cell receptor. For example, the population can include a plurality of genetically-modified eukaryotic cells of the invention which express a CAR (i.e., are CAR+), or an exogenous T cell receptor (i.e., exoTCR+), and do not exhibit expression of an endogenous alpha/beta T cell receptor (i.e., are TCR−). In various embodiments of the invention, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified eukaryotic cell as described herein. In a particular example, the population can comprise at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, cells that are both TCR− and positive for expression of the exogenous sequence of interest (e.g., CAR+ or exoTCR+).

In some embodiments, when introduced into a population of cells, the presently disclosed engineered meganucleases result in a greater percentage of the population of cells that are both TCR− and positive for expression of the exogenous sequence of interest (e.g., CAR+ or exoTCR+) than when the TRC 1-2L.1592 meganuclease is introduced into a population of cells.

Further, cells that have been genetically-modified with the presently disclosed engineered meganucleases exhibit improved characteristics, including reduced off-target cutting and effects thereof, and exhibit increased CAR T expansion, as compared to cells that have been genetically-modified with the TRC1-2L.1592 meganuclease.

### 2.4 Pharmaceutical Compositions

In some embodiments, the invention provides a pharmaceutical composition comprising a genetically-modified eukaryotic cell of the invention, or a population of genetically-modified eukaryotic cells of the invention, and a pharmaceutically-acceptable carrier. Such pharmaceutical compositions can be prepared in accordance with known techniques. See, e.g., Remington, The Science And Practice of Pharmacy (21st ed., Philadelphia, Lippincott, Williams & Wilkins, 2005). In the manufacture of a pharmaceutical formulation according to the invention, cells are typically admixed with a pharmaceutically acceptable carrier and the resulting composition is administered to a subject. The carrier must, of course, be acceptable in the sense of being compatible with any other ingredients in the formulation and must not be deleterious to the subject. In some embodiments, pharmaceutical compositions of the invention can further comprise one or more additional agents useful in the treatment of a disease in the subject. In additional embodiments, pharmaceutical compositions of the invention can further include biological molecules, such as cytokines (e.g., IL-2, IL-7, IL-15, and/or IL-21), which promote in vivo cell proliferation and engraftment of genetically-modified T cells. Pharmaceutical compositions comprising genetically-modified eukaryotic cells of the invention can be administered in the same composition as an additional agent or biological molecule or, alternatively, can be co-administered in separate compositions.

The present disclosure also provides genetically-modified eukaryotic cells, or populations thereof, described herein for use as a medicament. The present disclosure further provides the use of genetically-modified eukaryotic cells or populations thereof described herein in the manufacture of a medicament for treating a disease in a subject in need thereof. In one such aspect, the medicament is useful for cancer immunotherapy in subjects in need thereof.

The present disclosure also provides genetically-modified eukaryotic cells, or populations thereof, described herein for use as a medicament. The present disclosure further provides the use of genetically-modified eukaryotic cells or populations thereof described herein in the manufacture of a medicament for treating a disease in a subject in need thereof. In one such aspect, the medicament is useful for cancer immunotherapy in subjects in need thereof.

Pharmaceutical compositions of the invention can be useful for treating any disease state that can be targeted by cell adoptive immunotherapy. In a particular embodiment, the pharmaceutical compositions and medicaments of the invention are useful in the treatment of cancer. Non-limiting examples of cancer which may be treated with the pharmaceutical compositions and medicaments of the present disclosure are carcinomas, lymphomas, sarcomas, melanomas, blastomas, leukemias, and germ cell tumors, including but not limited to cancers of B-cell origin, neuroblastoma, osteosarcoma, prostate cancer, renal cell carcinoma, rhabdomyosarcoma, liver cancer, gastric cancer, bone cancer, pancreatic cancer, skin cancer, cancer of the head or neck, breast cancer, lung cancer, cutaneous or intraocular malignant melanoma, renal cancer, uterine cancer, ovarian cancer, colorectal cancer, colon cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, non-Hodgkin's lymphoma, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, environmentally induced cancers including those induced by asbestos, multiple myeloma, Hodgkin lymphoma, non-Hodgkin's lymphomas, acute myeloid lymphoma, chronic myelogenous leukemia, chronic lymphoid leukemia, immunoblastic large cell lymphoma, acute lymphoblastic leukemia, mycosis fungoides, anaplastic large cell lymphoma, and T-cell lymphoma, and any combinations of said cancers. In certain embodiments, cancers of B-cell origin include, without limitation, B-lineage acute lymphoblastic leukemia, B-cell chronic lymphocytic leukemia, B-cell lymphoma, diffuse large B cell lymphoma, pre-B ALL (pediatric indication), mantle cell lymphoma, follicular lymphoma, marginal zone lymphoma, Burkitt's lymphoma, multiple myeloma, and B-cell non-Hodgkin's lymphoma.

In some of these embodiments wherein cancer is treated with the presently disclosed genetically-modified eukaryotic cells or populations thereof, the subject administered the genetically-modified eukaryotic cells or populations thereof is further administered an additional therapeutic, such as radiation, surgery, or a chemotherapeutic agent.

The invention further provides a population of eukaryotic cells comprising a plurality of genetically-modified eukaryotic cells described herein, which comprise in their genome an exogenous nucleic acid molecule encoding a sequence of interest, wherein the exogenous nucleic acid molecule is inserted into the T cell receptor alpha constant region gene at the TRC 1-2 recognition sequence, and wherein expression of the endogenous alpha/beta TCR is eliminated. Thus, in various embodiments of the invention, a population of genetically-modified eukaryotic cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified eukaryotic cell described herein. In further embodiments of the invention, a population of genetically-modified cells is provided wherein at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or up to 100%, of cells in the population are a genetically-modified eukaryotic cell described herein which further express a protein encoded by the exogenous sequence of interest (e.g., a CAR or exoTCR).

### 2.5. Methods of Administering Genetically-Modified Cells

Another aspect disclosed herein is the administration of an effective amount of the genetically-modified eukaryotic cells, or populations thereof, of the present disclosure to a subject in need thereof. In particular embodiments, the pharmaceutical compositions described herein are administered to a subject in need thereof. For example, an effective amount of a population of cells can be administered to a subject having a disease. In particular embodiments, the disease can be cancer, and administration of the genetically-modified eukaryotic cells of the invention represent an immunotherapy. The administered cells are able to reduce the proliferation, reduce the number, or kill target cells in the recipient. Unlike antibody therapies, genetically-modified eukaryotic cells of the present disclosure are able to replicate and expand in vivo, resulting in long-term persistence that can lead to sustained control of a disease.

Examples of possible routes of administration include parenteral, (e.g., intravenous (IV), intramuscular (IM), intradermal, subcutaneous (SC), or infusion) administration. Moreover, the administration may be by continuous infusion or by single or multiple boluses. In specific embodiments, the agent is infused over a period of less than about 12 hours, 6 hours, 4 hours, 3 hours, 2 hours, or 1 hour. In still other embodiments, the infusion occurs slowly at first and then is increased over time.

In some embodiments, a genetically-modified eukaryotic cell, or population thereof, of the present disclosure targets a tumor antigen for the purposes of treating cancer such as, for example, those tumor antigens and cancers described elsewhere herein

When an “effective amount” or “therapeutic amount” is indicated, the precise amount to be administered can be determined by a physician with consideration of individual differences in age, weight, tumor size (if present), extent of infection or metastasis, and condition of the patient (subject). In some embodiments, a pharmaceutical composition comprising the genetically-modified cells or populations thereof described herein is administered at a dosage of 10⁴ to 10⁹ cells/kg body weight, including all integer values within those ranges. In further embodiments, the dosage is 10⁵ to 10⁶ cells/kg body weight, including all integer values within those ranges. In some embodiments, cell compositions are administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In some embodiments, administration of genetically-modified eukaryotic cells or populations thereof of the present disclosure reduce at least one symptom of a target disease or condition. For example, administration of genetically-modified T cells, or populations thereof, of the present disclosure can reduce at least one symptom of a cancer. Symptoms of cancers are well known in the art and can be determined by known techniques.

### 2.6 Engineered Nuclease Variants

Embodiments disclosed herein encompass the engineered meganucleases described herein, and variants thereof. Further embodiments of the invention encompass polynucleotides comprising a nucleic acid sequence encoding the meganucleases described herein, and variants of such polynucleotides.

As used herein, “variants” is intended to mean substantially similar sequences. A “variant” polypeptide is intended to mean a polypeptide derived from the “native” polypeptide by deletion or addition of one or more amino acids at one or more internal sites in the native protein and/or substitution of one or more amino acids at one or more sites in the native polypeptide. As used herein, a “native” polynucleotide or polypeptide comprises a parental sequence from which variants are derived. Variant polypeptides encompassed by the embodiments are biologically active. That is, they continue to possess the desired biological activity of the native protein; i.e., the ability to recognize and cleave the TRC 1-2 recognition sequence (SEQ ID NO: 5) found in the human T cell receptor alpha constant region (SEQ ID NO: 3), and in some embodiments, exhibit at least one improved property over previously developed TRC 1-2 meganucleases. Such variants may result, for example, from human manipulation. Biologically active variants of a native polypeptide of the embodiments (e.g., SEQ ID NOs: 7-10), or biologically active variants of the recognition half-site binding subunits described herein, will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, or about 99%, sequence identity to the amino acid sequence of the native polypeptide or native subunit, as determined by sequence alignment programs and parameters described elsewhere herein. A biologically active variant of a polypeptide or subunit of the embodiments may differ from that polypeptide or subunit by as few as about 1-40 amino acid residues, as few as about 1-20, as few as about 1-10, as few as about 5, as few as 4, 3, 2, or even 1 amino acid residue.

The polypeptides of the embodiments may be altered in various ways including amino acid substitutions, deletions, truncations, and insertions. Methods for such manipulations are generally known in the art. For example, amino acid sequence variants can be prepared by mutations in the DNA. Methods for mutagenesis and polynucleotide alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (MacMillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be optimal.

In some embodiments, engineered meganucleases of the invention can comprise variants of the HVR1 and HVR2 regions disclosed herein. Parental HVR regions can comprise, for example, residues 24-79 or residues 215-270 of the exemplified engineered meganucleases. Thus, variant HVRs can comprise an amino acid sequence having at least 80%, at least 85%, at least 90%, at least 91%, at least 92%, at least 93%, at least 94%, at least 95%, at least 96%, at least 97%, at least 98%, at least 99%, or more, sequence identity to an amino acid sequence corresponding to residues 24-79 or residues 215-270 of the engineered meganucleases exemplified herein, such that the variant HVR regions maintain the biological activity of the engineered meganuclease (i.e., binding to and cleaving the recognition sequence). Further, in some embodiments of the invention, a variant HVR1 region or variant HVR2 region can comprise residues corresponding to the amino acid residues found at specific positions within the parental HVR. In this context, “corresponding to” means that an amino acid residue in the variant HVR is the same amino acid residue (i.e., a separate identical residue) present in the parental HVR sequence in the same relative position (i.e., in relation to the remaining amino acids in the parent sequence). By way of example, if a parental HVR sequence comprises a serine residue at position 26, a variant HVR that “comprises a residue corresponding to” residue 26 will also comprise a serine at a position that is relative (i.e., corresponding) to parental position 26.

In particular embodiments, engineered meganucleases disclosed herein comprise an HVR1 region that has at least 90%, at least 91%, at least 92%, at least 93%, at least 94%, at least 95%, at least 96%, at least 97%, at least 98%, at least 99%, or more sequence identity to an amino acid sequence corresponding to residues 215-270 of any one of SEQ ID NOs: 7-10.

In certain embodiments, engineered meganucleases disclosed herein comprise an HVR2 region that has at least at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more sequence identity to an amino acid sequence corresponding to residues 24-79 of any one of SEQ ID NOs: 7-10.

A substantial number of amino acid modifications to the DNA recognition domain of the wild-type I-CreI meganuclease have previously been identified (e.g., U.S. Pat. No. 8,021,867) which, singly or in combination, result in recombinant meganucleases with specificities altered at individual bases within the DNA recognition sequence half-site, such that the resulting rationally-designed meganucleases have half-site specificities different from the wild-type enzyme. Table 3 provides potential substitutions that can be made in an engineered meganuclease monomer or subunit to enhance specificity based on the base present at each half-site position (−1 through −9) of a recognition half-site.

TABLE 3

| | Favored Sense-Strand Base | | | | | | | | | | |
|---|---|---|---|---|---|---|---|---|---|---|---|
| Posn. | A | C | G | T | A/T | A/C | A/G | C/T | G/T | A/G/T | A/C/G/T |

| | | | | | | | | | | |
|---|---|---|---|---|---|---|---|---|---|---|
| −1 | Y75 | R70* | K70 | Q70* | | | | T46* | | G70 |
| | L75* | H75* | E70* | C70 | | | | | | A70 |
| | C75* | R75* | E75* | L70 | | | | | | S70 |
| | Y139* | H46* | E46* | Y75* | | | | | | G46* |
| | C46* | K46* | D46* | Q75* | | | | | | |
| | A46* | R46* | | H75* | | | | | | |
| | | | | H139 | | | | | | |
| | | | | Q46* | | | | | | |
| | | | | H46* | | | | | | |
| −2 | Q70 | E70 | H70 | Q44* | C44* | | | | | |
| | T44* | D70 | D44* | | | | | | | |
| | A44* | K44* | E44* | | | | | | | |
| | V44* | R44* | | | | | | | | |
| | I44* | | | | | | | | | |
| | L44* | | | | | | | | | |
| | N44* | | | | | | | | | |
| −3 | Q68 | E68 | R68 | M68 | | H68 | | Y68 | K68 | |
| | C24* | F68 | | C68 | | | | | | |
| | I24* | K24* | | L68 | | | | | | |
| | | R24* | | F68 | | | | | | |
| −4 | A26* | E77 | R77 | | | | | S77 | | S26* |
| | Q77 | K26* | E26* | | | | | Q26* | | |
| −5 | | E42 | R42 | | | K28* | C28* | | | M66 |
| | | | | | | | Q42 | | | K66 |
| −6 | Q40 | E40 | R40 | C40 | A40 | | | | | S40 |
| | C28* | R28* | | I40 | A79 | | | | | S28* |
| | | | | V40 | A28* | | | | | |
| | | | | C79 | H28* | | | | | |
| | | | | I79 | | | | | | |
| | | | | V79 | | | | | | |
| | | | | Q28* | | | | | | |
| −7 | N30* | E38 | K38 | I38 | | | C38 | | | H38 |
| | Q38 | K30* | R38 | L38 | | | | | | N38 |
| | | R30* | E30* | | | | | | | Q30* |
| −8 | F33 | E33 | F33 | L33 | | R32* | R33 | | | |
| | Y33 | D33 | H33 | V33 | | | | | | |
| | | | | I33 | | | | | | |
| | | | | F33 | | | | | | |
| | | | | C33 | | | | | | |
| −9 | | E32 | R32 | L32 | | | | D32 | | S32 |
| | | | K32 | V32 | | | | I32 | | N32 |
| | | | | A32 | | | | | | H32 |
| | | | | C32 | | | | | | Q32 |
| | | | | | | | | | | T32 |

Bold entries are wild-type contact residues and do not constitute “modifications” as used herein. An asterisk indicates that the residue contacts the base on the antisense strand.

Certain modifications can be made in an engineered meganuclease monomer or subunit to modulate DNA-binding affinity and/or activity. For example, an engineered meganuclease monomer or subunit described herein can comprise a G, S, or A at a residue corresponding to position 19 of I-CreI or any one of SEQ ID NOs: 7-10 (WO 2009001159), a Y, R, K, or D at a residue corresponding to position 66 of I-CreI or any one of SEQ ID NOs: 7-10, and/or an E, Q, or K at a residue corresponding to position 80 of I-CreI or any one of SEQ ID NOs: 7-10 (U.S. Pat. No. 8,021,867).

For polynucleotides, a “variant” comprises a deletion and/or addition of one or more nucleotides at one or more sites within the native polynucleotide. One of skill in the art will recognize that variants of the nucleic acids of the embodiments will be constructed such that the open reading frame is maintained. For polynucleotides, conservative variants include those sequences that, because of the degeneracy of the genetic code, encode the amino acid sequence of one of the polypeptides of the embodiments. Variant polynucleotides include synthetically derived polynucleotides, such as those generated, for example, by using site-directed mutagenesis but which still encode a recombinant nuclease of the embodiments. Generally, variants of a particular polynucleotide of the embodiments will have at least about 40%, about 45%, about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to that particular polynucleotide as determined by sequence alignment programs and parameters described elsewhere herein. Variants of a particular polynucleotide of the embodiments (i.e., the reference polynucleotide) can also be evaluated by comparison of the percent sequence identity between the polypeptide encoded by a variant polynucleotide and the polypeptide encoded by the reference polynucleotide.

The deletions, insertions, and substitutions of the variant protein sequences encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by screening the polypeptide for its ability to preferentially recognize and cleave the TRC 1-2 recognition sequence (SEQ ID NO: 5) found within exon 1 of the human T cell receptor alpha constant region gene (SEQ ID NO: 3).

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Optimization of Engineered TRC 1-2 Meganucleases

### 1. Meganuclease Optimization and Selection

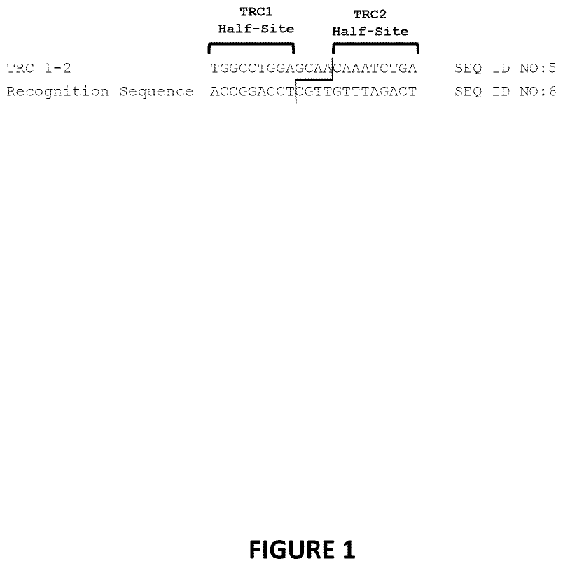

The goal of these studies was to develop engineered meganucleases capable of binding and cleaving the TRC 1-2 recognition sequence (SEQ ID NO: 5; FIG. 1) with improved characteristics relative to the TRC 1-2L.1592 meganuclease (SEQ ID NO: 24). Such improvements could include, for example, enhanced on-target activity and/or reduced off-target cleavage activity.

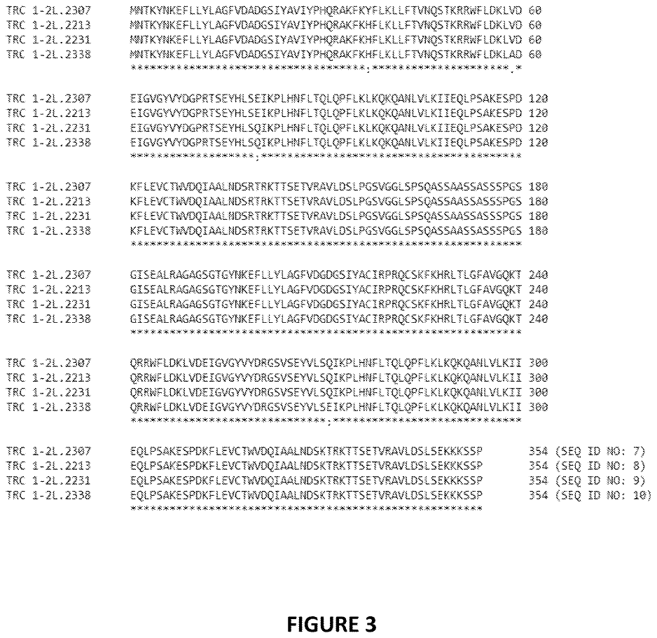

In two separate rounds of optimization, nuclease libraries were used to randomize key amino acids involved in recognition site specificity and DNA binding affinity of the TRC 1-2L.1592 meganuclease. In each optimization round, clones were selected and 96 well plates of individual answers were generated. These optimization rounds generated data that was combined to increase total specificity in an additional optimization round, in which specific amino acids in the TRC 1-2L.1592 meganuclease were randomized in an effort to enhance on-target activity while maintaining or improving the off-targeting profile. In this third round of optimization, clones were again selected following use of nuclease libraries for randomization, and a 96 well plate of individual answers was generated. Among the answers generated, the following optimized meganucleases were selected for further evaluation: TRC 1-2L.2307 (SEQ ID NO: 7), TRC 1-2L.2213 (SEQ ID NO: 8), TRC 1-2L.2231 (SEQ ID NO: 9), and TRC 1-2L.2338 (SEQ ID NO: 10). An alignment of these four TRC 1-2 meganucleases is shown in FIG. 3.

### 2. Oligo Capture Analysis of Off-Targeting

Each of the four selected optimized TRC 1-2 meganucleases was evaluated for off-target cleavage activity relative to the parental TRC 1-2L.1592 meganuclease, and the early-generation TRC 1-2x.878EE meganuclease (SEQ ID NO: 23).

Off-targeting was evaluated using an oligo capture assay. Similar to GUIDE-seq, the oligo capture assay identifies potential off-target sites produced by the TRC 1-2 meganucleases by capturing an oligonucleotide at break sites within the cell's genomic DNA. GUIDE-seq was developed for CRISPR-Cas9 generated DNA breaks and there are a few key modifications to the chemistry and analysis in order to apply this technique to the present nucleases. Unlike CRISPR-Cas9, the engineered meganucleases of the invention generate a four base pair 3′ overhang. To accommodate for this difference, the oligonucleotides used in oligo capture have randomized four base pair overhangs that could be compatible with the overhangs generated with the TRC 1-2 meganuclease. A higher frequency of insertion is observed due to the greater efficiency of ligating sticky ends rather than blunt ends. T cells were transfected with mRNA encoding individual TRC 1-2 meganucleases and the double stranded DNA oligonucleotides. After two days, genomic DNA from these cells was isolated and sonicated to shear the DNA to smaller sizes. An oligonucleotide adapter was ligated to the sheared DNA and PCR was used to amplify any DNA pieces that contain an adapter at one end and the captured oligonucleotide at the other end. The amplified DNA was purified and sequencing libraries were prepared using standard commercial kits.

The libraries were sequenced for paired end 150 bp reads on an Illumina sequencer. The data was filtered and analyzed for valid oligonucleotide integrations and the location of the potential off-target site is calculated. Here again, the protocol needed to be adjusted from the PAM search used for CRISPR-Cas9 to the TRC 1-2 meganuclease search. The software developed checks each sequence to make sure there is adapter and captured oligo flanking the sequence to verify that it is a valid read. The software also checks for PCR duplicates and removes reads that are identical to help reduce PCR bias. The sequence reads are aligned to a reference genome and grouped sequences within thousand base pair windows are scanned for a potential TRC 1-2 meganuclease site.

Each TRC 1-2 meganuclease is a linked dimer. Each monomer recognizes a nine-base pair half site with a four base pair spacer in the center between the two half sites. The software looks for the closest sequence match for each half site with no allowed gaps. The middle four base pairs are not considered in the off-target selection because the TRC 1-2 meganucleases can generally tolerate a higher amount of degeneracy at these positions in the target site. The software outputs a list of potential off-target sites with the number of base mismatches in the combined half sites but not counting the middle four base pair mismatches. The software does not eliminate any off-targets based on an arbitrary mismatch filter, unlike GUIDE-Seq which eliminates any off-target identified with more than six base pairs mismatched. Instead, background noise generated from random capture of the oligo at fragile spots or hot spots within the genome can be reduced in two ways. First, an untreated mock sample is also run though oligo capture and windows of integration sites without the nuclease present can be subtracted from the nuclease containing samples. We have also found that running the assay in triplicate and eliminating any sites that do not repeat in at least two of the three repeats is a good way to empirically remove random integration noise.

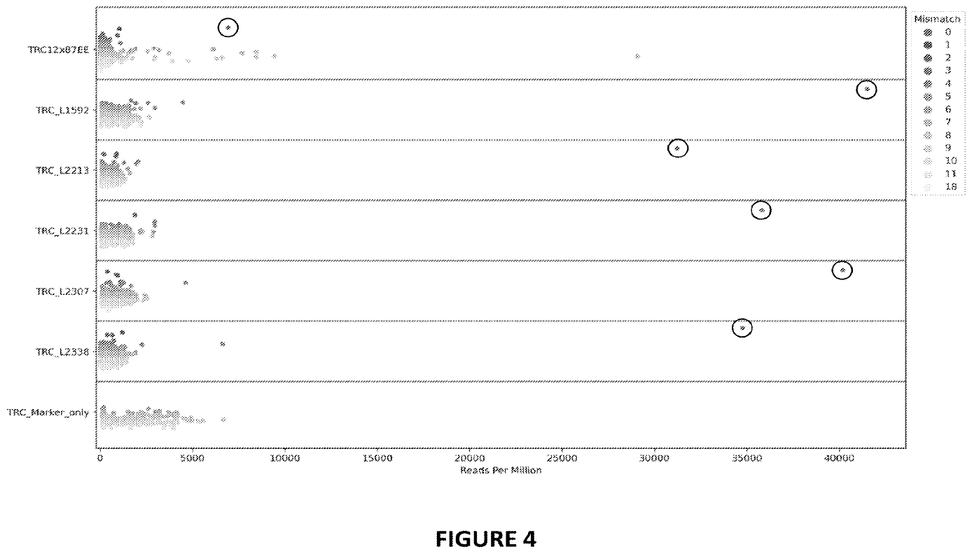

Although read count does not directly correlate with cutting frequency at a particular site, it can generally highlight off-targets that are more probable to be a verified off-target site because they occur more often. Additional visualization methods enable us to look at the oligo capture data not only in terms of number of reads recovered at a particular site, but also by number of mismatches between a putative off-target site and the intended site. This allows for a more accurate determination of real oligo integrations sites as compared to random integration or sequencing noise. In FIG. 4, off target sites are plotted according to their number of aligned reads on the X axis (normalized to reads at each site per million reads sequenced), and the number of mismatched base pairs compared to the intended site are indicated by color, with darker colors indicating closer overall matches between off-targets and the intended binding site. The intended target site for each sample is identified with a circle.

FIG. 4 illustrates that the four optimized TRC 1-2 meganucleases from the latest round of optimization, and the TRC 1-2L.1592 meganuclease, exhibit substantially fewer off-target cleavage sites relative to the early generation TRC 1-2x.87 EE meganuclease, with decreases in the number of higher read-count sites and decreases in sites more similar to the intended. Moreover, each of the four optimized TRC 1-2 meganucleases maintained, or improved upon, the off-targeting profile of TRC 1-2L.1592. Accordingly, all four optimized meganucleases were further evaluated for on-target activity relative to the parental TRC 1-2L.1592.

### Example 2

### On-Target Activity of Engineered TRC 1-2 Meganucleases in Primary Human T Cells

### 1. Materials and Methods

The purpose of this study was to evaluate the on-target activity of optimized TRC 1-2 meganucleases relative to the parental TRC 1-2L.1592 meganuclease.

For this study, an apheresis sample was drawn from a healthy, informed, and compensated donor, and the T cells were enriched using the CD3 positive selection kit II in accord with the manufacturer's instructions (Stem Cell Technologies). T cells were activated using ImmunoCult T cell stimulator (anti-CD2/CD3/CD28—Stem Cell Technologies) in X-VIVO 15 medium (Lonza) supplemented with 5% fetal bovine serum and 10 ng/ml IL-2 (Gibco). After 3 days of stimulation, cells were collected and samples of 1e6 cells were electroporated with 1000 ng (high dose) or 300 ng (low dose) of RNA encoding a meganuclease that recognizes and cleaves the TRC 1-2 sequence in the T cell receptor alpha constant locus. Five different TRC 1-2 meganucleases were studied in this experiment: TRC 1-2L.1592 (benchmark), TRC 1-2L.2213, TRC 1-2L.2231, TRC1-2L.2307, and TRC 1-2L.2338.

Following electroporation, cells were cultured in complete X-VIVO 15 supplemented with 30 ng/ml recombinant human IL-2 for 7 days with medium exchanges occurring every 2-3 days. On day 7, cells were counted and a sample from each culture was harvested and stained with anti-CD3-BV421 (BioLegend). Data were acquired on a Beckman-Coulter CytoFLEX flow cytometer. A table of the knockout frequency and overall number of knockouts generated for a given dose of nuclease appears in the results section below.

### 2 Results

In this study, the benchmark TRC 1-2L.1592 meganuclease demonstrated an editing rate of 40% at the high RNA dose and 7% editing at the low RNA dose. It was observed that all four new nucleases demonstrated a higher editing frequency at the high dose of RNA than the benchmark, but displayed less of a reduction when the low dose of RNA was delivered (see Table 4 below). Notably, the TRC 1-2L.2213 and TRC 1-2L.2307 meganucleases supported superior production of TRAC-edited T cells at the 300 ng low dose than TRC 1-2L.1592 meganuclease supported at the 1000 ng high dose.

TABLE 4

| | | | D 0 | |
|---|---|---|---|---|
| | | Total edited | Total | TRC KO cells/ |
| Sample | KO % | cells | Cells | input cell |
| 1592 (1000 ng) | 40.87% | 3.45e6 | 6.30e5 | 5.47 |
| 1592 (300 ng) | 7.81% | 6.07e5 | 5.80e5 | 1.05 |
| 2213 (1000 ng) | 63.78% | 5.86e6 | 6.75e5 | 8.69 |
| 2213 (300 ng) | 42.99% | 3.62e6 | 4.50e5 | 8.05 |
| 2231 (1000 ng) | 61.46% | 5.51e6 | 7.63e5 | 7.23 |
| 2231 (300 ng) | 39.24% | 2.55e6 | 7.48e5 | 3.42 |
| 2307 (1000 ng) | 66.00% | 4.78e6 | 5.88e5 | 8.13 |
| 2307 (300 ng) | 46.11% | 3.60e6 | 5.73e5 | 6.28 |
| 2338 (1000 ng) | 68.73% | 5.22e6 | 5.13e5 | 10.18 |
| 2338 (300 ng) | 37.16% | 2.75e6 | 6.75e5 | 4.08 |

### 3. Conclusions

In this experiment, all four optimized TRC 1-2 meganucleases outperformed the TRC 1-2L.1592 benchmark in a study where TRAC-edited T cell production was measured. These results supported further characterization of these the candidate meganucleases. Their relative abilities to produce CAR T cells, and the function of those CAR T cells, were further assessed.

### Example 3

### Assessment of CAR T Cells Manufactured Using Optimized TRC 1-2 Meganucleases

### 1. Materials and Methods

The purpose of this study was to evaluate the optimized TRC 1-2 meganucleases for their ability to generate CAR T cells having a CAR-coding sequence inserted in the TRAC locus at the TRC 1-2 recognition sequence. Six different TRC 1-2 nucleases were studied in this experiment: TRC 1-2x.87 EE (early-generation benchmark 1), TRC 1-2L.1592 (benchmark 2), TRC 1-2L.2213, TRC 1-2L.2231, TRC1-2L.2307, and TRC1-2L.2338.

In this study, an apheresis sample was drawn from a healthy, informed, and compensated donor, and the T cells were enriched using the CD3 positive selection kit II in accord with the manufacturer's instructions (Stem Cell Technologies).

T cells were processed according to the following protocol: T cell enrichment using human CD3 positive selection reagents (StemCell Technologies), stimulation using ImmunoCult anti-CD2/CD3/CD28 (StemCell Technologies) and nuclease RNA delivery using the 4D NucleoFEctor (Lonza). Three days after initial stimulation with ImmunoCult/TransAct, T cells were harvested, electroporated with 1 μg per 1×10⁶ cells of RNA encoding one of the TRC 1-2 meganucleases, and immediately transduced with an AAV6 vector encoding an anti-CD19 CAR gene to be inserted into the TRC 1-2 recognition sequence following nuclease cleavage.

Cell counts were acquired for each culture using a Countess II automated cell counter (Thermo-Fisher) and a sample was stained with anti-CD3-BV421 (BioLegend) and anti-FMC63-Alx647 (BioLegend) on day 5 post-electroporation. Data were acquired on a Beckman-Coulter CytoFLEX flow cytometer.

Non edited cells were removed by magnetic depletion (StemCell Technologies CD3 selection kit II) and culture for an additional 2 days in 10 ng/ml each of IL-15 and IL-21 (Gibco). Following this, cells were counted and plated in a functional assay. CAR T cells produced using each TRC 1-2 meganuclease were co-cultured with K562 cells engineered to stably express CD19 at effector-to-target ratios of 1:1, 1:3, and 1:9 where 1=2×10⁴ cells. Medium was refreshed after 2 days and samples of the culture were taken for analysis after 5 days. Samples were stained with anti-CD4-APC, anti-CD8-FITC, and anti-CD19-PE and data were acquired on a Beckman-Coulter CytoFLEX cytometer which enables absolute cell enumeration. The number of resulting T cells and surviving target cells were captured and used to calculate T cell expansion and target cell killing percentage.

### 2. Results

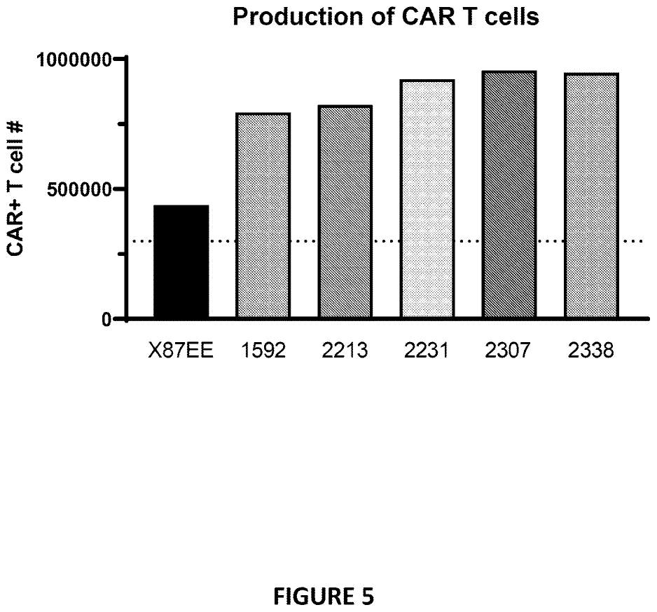

Using cell counts and frequency of TRAC-edited CAR+ events, the number of CAR T cells produced using each TRC 1-2 meganuclease variant were calculated and plotted in FIG. 5. TRC 1-2x.87 EE produced the fewest CAR T cells while the TRC 1-2L.2231, TRC 1-2L.2307, and TRC 1-2L.2338 meganucleases produced more CAR T cells than the TRC 1-2L.1592 benchmark.

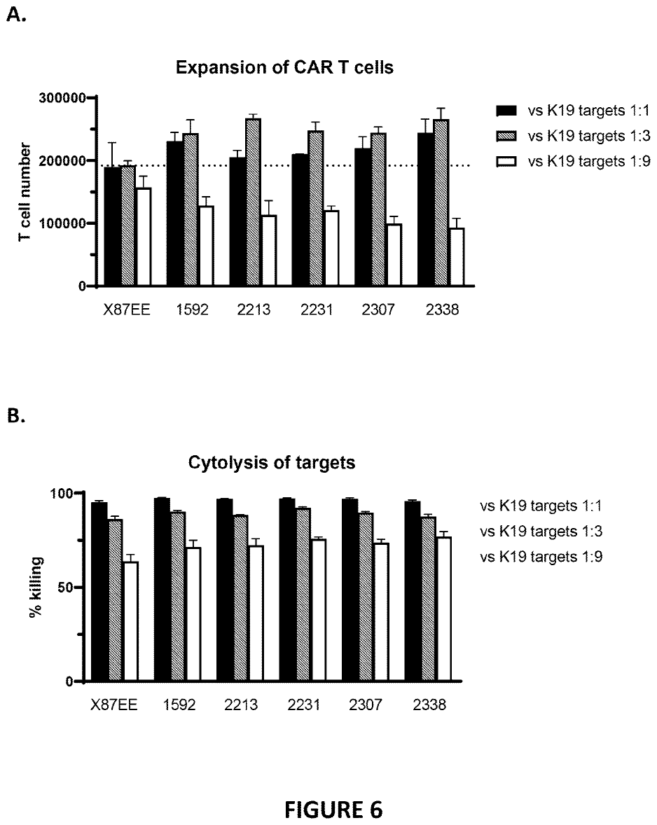

Assessment of CAR T proliferation and target cell killing following antigen engagement did not reveal any differences between the CAR T preparations except for the observation that cells produced using TRC 1-2x.87EE exhibited less proliferation (FIG. 6A) compared to all other CAR T batches. No differences were observed in terms of target cytolysis (FIG. 6B).

### 3 Conclusions

In this study, three of the candidate optimized TRC 1-2 meganucleases outperformed the TRC 1-2L.1592 benchmark in terms of CAR T cell production (L.2231, L.2307, and L.2338), with the TRC 1-2L.2307 meganuclease generating the highest number.

### Example 4

### Determination of EC50 and EC90 of Optimized TRC 1-2 Meganucleases

### 1 Materials and Methods

The purpose of this study was to determine the EC50 and EC90 values of the optimized TRC 1-2L.2307 meganuclease, and to compare these values to the benchmark TRC 1-2L.1592 meganuclease.

In this study, an apheresis sample was drawn from a healthy, informed, and compensated donor, and the T cells were enriched using the CD3 positive selection kit II in accord with the manufacturer's instructions (Stem Cell Technologies). T cells were prepared for editing by stimulating with ImmunoCult anti-CD3/CD28/CD2 (Stem Cell Technologies) for three days in Xuri medium (GE Healthcare) supplemented with fetal bovine serum to 5% (Gemini Bio) and recombinant human IL-2 to 10 ng/ml (Gibco). Stimulated T cells were harvested and counted (Countess II, ThermoFisher) before electroporation was carried out on the Lonza 4-D NucleoFector using a titrated range of mRNA encoding TRC 1-2L.1592 or TRC 1-2L.2307. The concentrations used for this study were 2, 1, 0.5, 0.25, 0.125, and 0.0625 μg of RNA per 1×10⁶ stimulated T cells. Cells were subsequently cultured as above with IL-2 supplemented to 30 ng/ml.

Three days following nucleofection, samples of each culture were withdrawn and stained with antibodies against CD4, CD8, and CD3. The frequency of CD3-(TRAC knockout) cells was plotted against RNA added to each reaction using GraphPad Prism software.

### 2. Results

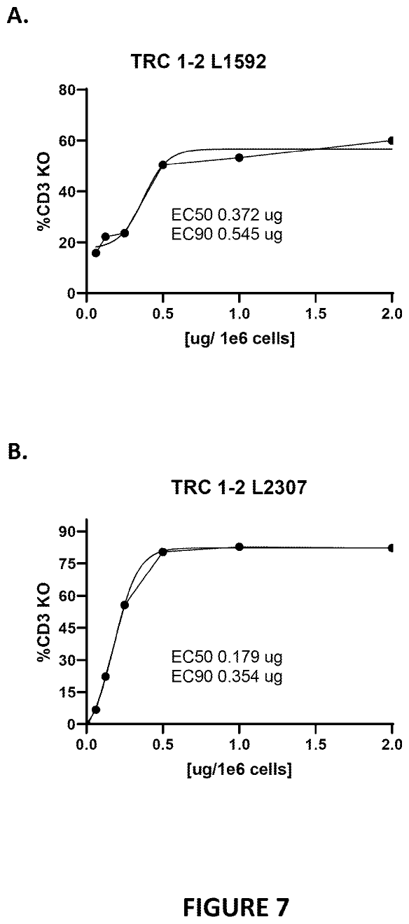

Using cell counts and frequency of TRAC-edited CAR+ events, the number of CAR T cells produced using each TRC1-2 variant were calculated and plotted in FIG. 7. GraphPad Prism was used to calculate EC50 and EC90 values for TRC 1-2L.1592 (FIG. 7A), and TRC 1-2L.2307 (FIG. 7B). The EC50 and EC90 values for TRC 1-2L.1592 were 0.372 and 0.545 μg, respectively. By comparison, the EC50 and EC90 values of the optimized TRC 1-2L.2307 meganuclease were 0.179 and 0.354 μg, respectively.

### 3. Conclusions

The optimized nuclease TRC 1-2L.2307 demonstrated substantially higher potency than the TRC 1-2L.1592 benchmark in this comparison, as evidenced by lower EC50 and EC90 values.

### Example 5

### Evaluation of Knock-In/Knockout Frequency of Optimized TRC 1-2 Meganucleases

### 1 Materials and Methods

The purpose of this study was to evaluate the characteristics of CAR T cells generated using the TRC 1-2L.1592 benchmark versus the optimized TRC 1-2L.2307 meganuclease, particularly the knock-in/knockout frequencies at the TRAC locus.

In this study, an apheresis sample was drawn from a healthy, informed, and compensated donor, and the T cells were enriched using the CD3 positive selection kit II in accord with the manufacturer's instructions (Stem Cell Technologies). T cells were prepared for editing by stimulating with ImmunoCult anti-CD3/CD28/CD2 (Stem Cell Technologies) for three days in Xuri medium (GE Healthcare) supplemented with fetal bovine serum to 5% (Gemini Bio) and recombinant human IL-2 to 10 ng/ml (Gibco). Stimulated T cells were harvested and counted (Countess II, ThermoFisher) before electroporation was carried out on the Lonza 4-D NucleoFector using 500 ng of mRNA encoding TRC 1-2L.1592 or 300 ng TRC 1-2L.2307 per 1×10⁶ stimulated T cells. These are the approximate EC90 values determined for these nuclease RNAs in a previous study. Immediately following nucleofection, cells were transduced with 20,000 AAV6 genomes per cell to provide the CD19 CAR coding template and were subsequently cultured as above with IL-2 supplemented to 30 ng/ml.

Four days following nucleofection, samples of each culture were withdrawn and stained with antibodies against the anti-CD19 FMC63 CAR, and CD3 data were collected on a Beckman-Coulter CytoFLEX-LX cytometer.

### 2. Results

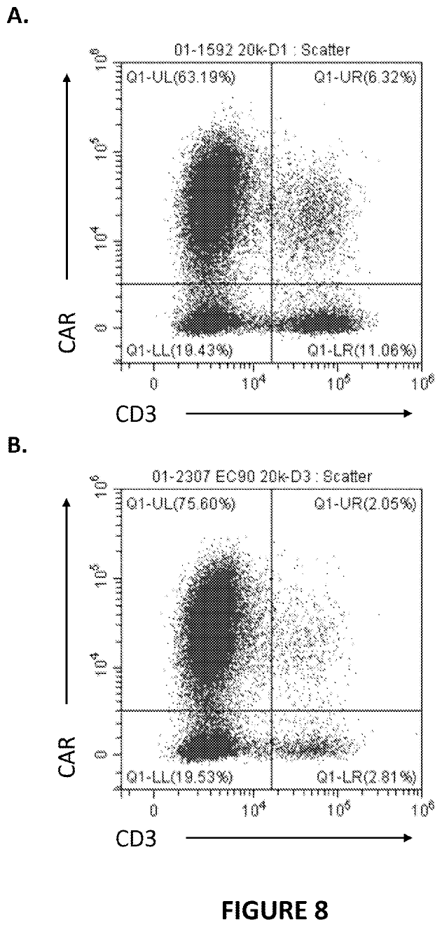

As shown in FIG. 8, cells edited using TRC 1-2L.1592 (FIG. 8A) demonstrated an overall TRAC knockout rate of 82%, wherein 63% of the overall culture was TRC−/CAR+, corresponding to a transgene insertion occurring in approximately 75% of edited cells. As shown in panel FIG. 8B, cells edited with TRC 1-2L.2307 demonstrated a knockout rate of 95% with 75% of the total culture displaying a CD3−/CAR+ phenotype. This corresponds to a transgene insertion occurring in approximately 79% of edited cells.

### 3. Conclusions

The TRC 1-2L.2307 meganuclease produced a substantially higher frequency of edited cells than the TRC 1-2L.1592 benchmark meganuclease, leading to an increase in the frequency of TRAC-edited CAR T cells.

### Example 6

### Effect of mRNA Optimization on TRC 1-2 Meganuclease Activity

### 1 Methods and Materials

This experiment was conducted to compare the efficiency of the TRC 1-2L.2307 meganuclease when encoded and delivered by a standard mRNA construct compared to an optimized mRNA construct in primary human T cells.

In this pair of studies, an apheresis sample was drawn from a healthy, informed, and compensated donor, and the T cells were enriched using the CD3 positive selection kit II in accordance with the manufacturer's instructions (Stem Cell Technologies). T cells were activated using ImmunoCult T cell stimulator (anti-CD2/CD3/CD28-Stem Cell Technologies) in Xuri medium (Cytiva) supplemented with 5% fetal bovine serum and 10 ng/ml IL-2 (Gibco). After 3 days of stimulation, cells were collected and electroporated with standard mRNA formulation of the TRC1-2 L.2307 meganuclease that recognizes and cleave the TRC 1-2 site or a novel optimized formulation (referred to as the “Max” formulation). The standard formulation was delivered in 2-fold titrations from 3540 ng per 1e6 cells down to 13.8 ng per 1e6 cells. The MAX formulation was delivered in 2-fold titrations from 4000 ng per 1e6 cells down to 62.5 ng per 1e6 cells.

Following electroporation, cells were cultured in complete Xuri supplemented with 30 ng/ml recombinant human IL-2 for 3-5 days with medium exchanges occurring every 2-3 days. Cells were counted after at least 3 days of culture, and stained for CD3 either by APC-conjugated anti-CD3 antibody (Biolegend) or FITC-conjugated anti-CD3 antibody (BioLegend). Data were acquired on a Beckman-Coulter CytoFLEX flow cytometer.

The DNA sequence of the constructs utilized in these experiments are provided in Table 5 below.

TABLE 5

| | | | N-term | | C-term | |
|---|---|---|---|---|---|---|
| mRNA Name | Promoter | 5′ UTR | NLS | Meganuclease | NLS | 3′ UTR |
| TRC 1-2L.2307 | T7AG | HBA2 | SV40 | TRC1-2L.2307 | — | WPRE |
| Standard mRNA | | | | | | |
| TRC1-2L.2307 | T7AG | ALB | SV40 | TRC1-2L.2307 | SV40 | SNRPBV1 |
| Optimized mRNA | | | | | | |

As shown, the standard mRNA construct includes, from 5′ to 3′, a T7AG promoter, an HBA2 5′ UTR, an N-terminal SV40 NLS, a coding sequence for the TRC 1-2L.2307 meganuclease, and a 3′ WPRE UTR. The optimized Max mRNA construct includes, from 5′ to 3′, a T7AG promoter, an ALB 5′ UTR, an N-terminal SV40 NLS, a coding sequence for the TRC 1-2L.2307 meganuclease, a C-terminal SV40 NLS, and a 3′ SNRBPV1 UTR.

### 2. Results

Successful targeting of the TRAC gene at the TCR 1-2 recognition site results in a loss of CD3 expression resulting in CD3 knock out (KO) cells. Table 6 below provides the knockout frequencies for the various experimental conditions.

TABLE 6

| | | # of KO | | |
|---|---|---|---|---|
| Sample | KO % | cells | Input | Fold Yield |
| L2307 3540 ng | 82.49% | | | |
| L2307 1760 ng | 82.2% | | | |
| L2307 884 ng | 82.15% | | | |
| L2307 442 ng | 78.455% | | | |
| L2307 221 ng | 57.46% | | | |
| L2307 111 ng | 27.165% | | | |
| L2307 55 ng | 7.835% | | | |
| L2307 27.6 ng | 8.09% | | | |
| L2307 13.8 ng | 4.37% | | | |
| L2307 Mock | 4.19% | | | |
| L2307 Max 4000 ng | 82.29% | 1.21e6 | 1e6 | 1.21 |
| L2307 Max 2000 ng | 89.32% | 2.33e6 | 1e6 | 2.33 |
| L2307 Max 1000 ng | 88.76% | 7.39r6 | 1e6 | 7.39 |
| L2307 Max 500 ng | 88.52% | 1.12e7 | 1e6 | 11.2 |
| L2307 Max 250 ng | 85.87% | 1.24e7 | 1e6 | 12.43 |
| L2307 Max 125 ng | 78.61% | 1.11e7 | 1e6 | 11.07 |
| L2307 Max 62.5 ng | 57.67% | 8.91e6 | 1e6 | 8.91 |
| L2307 Max Mock | 1.04% | NA | 1e6 | NA |

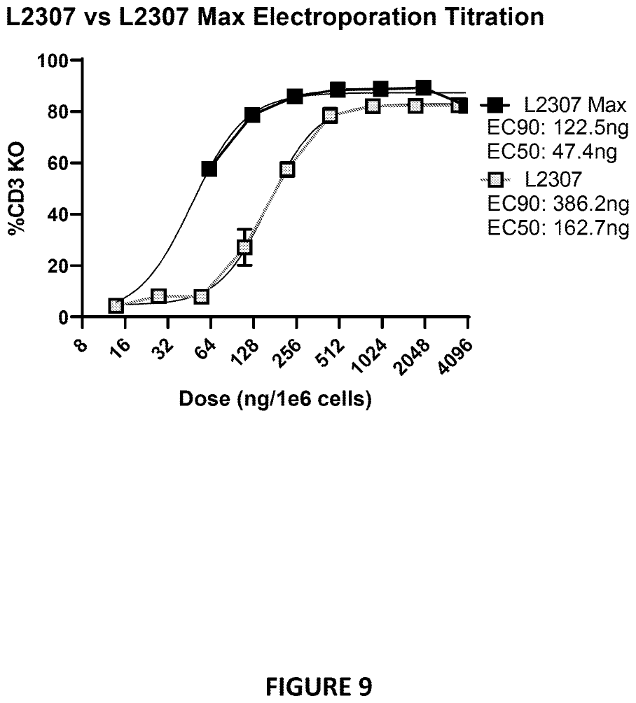

A dose response curve of CD3 knock out at various doses of the TRC1-2L.2307 meganuclease is provided in FIG. 9 with EC90 and EC50 doses for each titration curve. In this dose response curve, the standard mRNA and the Max mRNA encoding the TRC 1-2L.2307 meganuclease was compared. These mRNAs were delivered in 2-fold doses by electroporation. As shown, the Max mRNA reduced the EC90 and EC50 dose of the TRC1-2 L.2307 meganuclease by at least half. For example, electroporation of 125 ng of the Max mRNA encoding the TRC 1-2L.2307 meganuclease knocked out CD3 in 78.61% of T cells compared to the 78.45% generated by 442 ng of standard mRNA encoding the TRC 1-2L.2307 meganuclease.

### 3. Conclusions

The results of this experiment demonstrate that the optimized Max mRNA encoding the TRC 1-2L.2307 meganuclease outperformed a standard mRNA in a study where TRAC-edited T cell knockout-frequency was measured via CD3 knock out. These results demonstrate that the optimized Max mRNA encoding an engineered meganuclease performs in a superior fashion in a direct comparison to standard mRNA constructs.

1. An engineered meganuclease that binds and cleaves a recognition sequence comprising SEQ ID NO: 5 in a T cell receptor alpha constant region (TRAC) gene, wherein said engineered meganuclease comprises a first subunit and a second subunit, wherein said first subunit binds to a first recognition half site of said recognition sequence and comprises a first hypervariable (HVR1) region, and wherein said second subunit binds to a second recognition half site of said recognition sequence and comprises a second hypervariable (HVR2) region, wherein said engineered meganuclease comprises an amino acid sequence of any one of SEQ ID NOs: 7-10.

2-26. (canceled)

27. The engineered meganuclease of claim 1, wherein said engineered meganuclease is encoded by a nucleic acid sequence set forth in any one of SEQ ID NOs: 19-22.

28-116. (canceled)
